(19) United States
(12) Patent Application Publication
(10) Pub. No.: US 2026/0253708 A1
(43) Pub. Date: Aug. 27, 2026

(54) SYSTEM

(71) Applicant: SoftBank Group Corp., Tokyo (JP)

(72) Inventor: Koji KURONUMA, Tokyo (JP)

(73) Assignee: SoftBank Group Corp., Tokyo (JP)

(21) Appl. No.: 19/536,221

(22) Filed: Feb. 11, 2026

(30) Foreign Application Priority Data
Feb. 21, 2025 (JP) 2025-027067

(51) Int. Cl. G16H 20/60; G06F 1/16

(52) U.S. Cl. CPC G16H 20/60; G06F 1/163

(57) ABSTRACT

The system according to the embodiment comprises a receiving unit, a proposal unit, an acquisition unit, an accumulation unit, and an identification unit. The receiving unit receives allergy information, food preferences, or information on an amount of exercise of a user. The proposal unit analyzes the information received by the receiving unit and proposes a recipe. The acquisition unit acquires special sale information of a nearby supermarket. The accumulation unit accumulates ingredient list images of consumed food. The identification unit identifies which ingredient is suspected when an allergic symptom occurs.

### CROSS-REFERENCE TO RELATED APPLICATION

The present application claims priority to and incorporates by reference the entire contents of Japanese Patent Application No. 2025-027067 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, recipe proposals considering allergies, food preferences, and an amount of exercise have not been sufficiently performed, and there is room for improvement.

### SUMMARY OF THE INVENTION

The system according to the embodiment comprises a receiving unit, a proposal unit, an acquisition unit, an accumulation unit, and an identification unit. The receiving unit receives allergy information, food preferences, or information on an amount of exercise of a user. The proposal unit analyzes the information received by the receiving unit and proposes a recipe. The acquisition unit acquires special sale information of a nearby supermarket. The accumulation unit accumulates ingredient list images of consumed food. The identification unit identifies which ingredient is suspected when an allergic symptom occurs.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

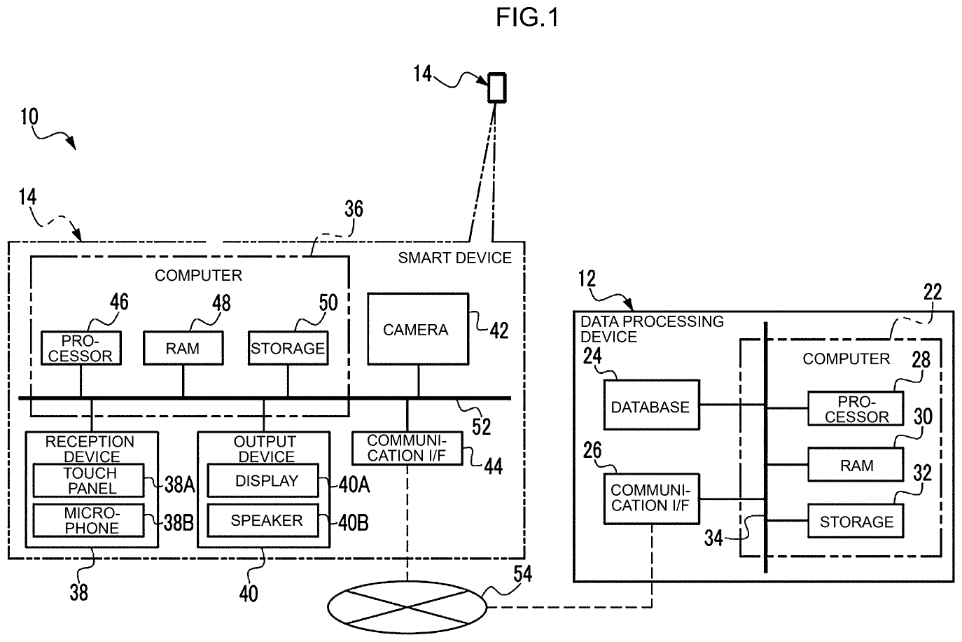

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

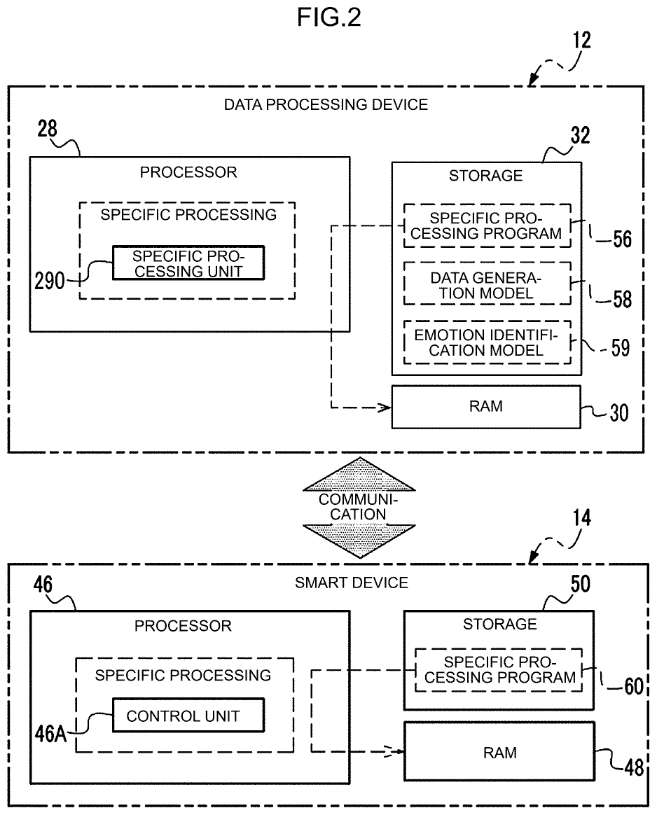

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

A recipe proposal system according to an embodiment of the present invention is a system that proposes a recipe considering allergy information, food preferences, an amount of exercise, and the like of a user. This recipe proposal system starts with the user inputting information such as allergy information of a child or the like, food preferences, and the amount of exercise. Next, an AI analyzes this information and proposes an appropriate recipe. In addition, the system acquires special sale information of a nearby supermarket and proposes a recipe utilizing profitable and seasonal ingredients. Furthermore, the system accumulates ingredient list images of consumed food, and when an allergic symptom occurs, the AI proposes which ingredient is suspected. This service is provided by an application on a smartphone or a tablet, and a user having a specific ID can use it for free. An advertisement is displayed at the bottom layer of a recipe page to secure revenue. For example, the user inputs information such as an allergy to a specific food, likes and dislikes, and a daily amount of exercise. This information is input to the AI. Next, the AI analyzes the input information and proposes an appropriate recipe. The AI generates a healthy and balanced recipe in consideration of the allergy information, the food preferences, the amount of exercise, and the like. For example, the AI proposes a recipe that excludes a food to which the user has an allergy and considers calories according to the amount of exercise. Furthermore, the system acquires special sale information of a nearby supermarket and proposes a recipe utilizing profitable and seasonal ingredients. The AI analyzes the special sale information and generates an economical and highly nutritious recipe. For example, the AI proposes a recipe using seasonal vegetables or meat on special sale. In addition, the system accumulates ingredient list images of consumed food, and when an allergic symptom occurs, the AI proposes which ingredient is suspected. When the user uploads an ingredient list image of the consumed food to the application, the AI analyzes the ingredients and identifies an ingredient that may cause an allergy. This service is provided by an application on a smartphone or a tablet, and a user having a specific ID can use it for free. For example, a user having an ID of a specific electronic payment system or messenger application can use the service for free. Finally, an advertisement is displayed at the bottom layer of the recipe page to secure revenue. Since the advertisement is displayed based on an interest or concern of the user, effective monetization is possible. Thereby, the recipe proposal system can propose a recipe considering the allergy information, the food preferences, the amount of exercise, and the like of the user, and perform accumulation of the special sale information and the ingredient list images, and identification of an allergic ingredient. Specifically, the present system adopts a distributed processing architecture in which a parallel computing server group on a cloud and a user terminal are connected via a secure communication line, and has a configuration for processing multi-dimensional lifelog data and biological information transmitted from the user terminal in real time. An AI processing unit in the present system is equipped with a deep learning model (for example, a large language model adopting a Transformer architecture or a convolutional neural network for image recognition), and accepts, as input data, a one-hot vector indicating user's allergy items, a preference feature vector representing food preferences, and time-series exercise data (a numerical array including the number of steps, heart rate, consumed calories, etc.) acquired from a wearable device or the like. The present AI processing unit maps these heterogeneous mixed data to an integrated high-dimensional feature space, executes similarity calculation (cosine similarity, etc.) with each recipe vector in a recipe database, thereby scoring a recipe optimal for a current context of the user, and outputs a high-ranked recipe. In the analysis of the special sale information, the present system extracts ingredient names and price information from POS data or Web flyer data of each supermarket using natural language processing, collates them with a nutritional value database, and executes an algorithm for solving an optimization problem that maximizes cost versus nutritional value (CP ratio). In the analysis of the ingredient list images, the present system takes image data captured by the user (for example, a high-resolution image tensor having a pixel array of 8 bits for each of RGB) as input, extracts raw material names as text data using optical character recognition (OCR) and an object detection model, and collates them with a known allergen database, thereby identifying an ingredient with a high allergy induction probability and outputting it together with a confidence score. These processing results are visualized in an application layer of the user terminal and displayed as a recipe proposal card or an allergen warning popup, and an advertisement delivery module performs real-time bidding (RTB) based on an interest/concern vector of the user to execute bidding for an optimal advertisement slot and display control.

The recipe proposal system according to the embodiment comprises a receiving unit, a proposal unit, an acquisition unit, an accumulation unit, and an identification unit. The receiving unit receives information such as allergy information, food preferences, and an amount of exercise of a user. The information input by the user includes, for example, an allergy to a specific food, likes and dislikes, a daily amount of exercise, and the like, but is not limited to such examples. The receiving unit enables the user to input information through, for example, an application on a smartphone or a tablet. The proposal unit analyzes the information received by the receiving unit and proposes an appropriate recipe. The proposal unit generates a healthy and balanced recipe considering the allergy information, the food preferences, the amount of exercise, and the like using, for example, AI. For example, the proposal unit proposes a recipe that excludes a food to which the user has an allergy and considers calories according to the amount of exercise. The acquisition unit acquires special sale information of a nearby supermarket. The acquisition unit collects and analyzes special sale information of a nearby supermarket through, for example, the Internet. For example, the acquisition unit analyzes the special sale information and generates an economical and highly nutritious recipe. The accumulation unit accumulates ingredient list images of consumed food. The accumulation unit uploads, for example, an ingredient list image of food consumed by the user to the application and analyzes ingredients thereof. For example, the accumulation unit stores the ingredient list image in a database and uses it to identify an ingredient that may cause an allergy. The identification unit identifies which ingredient is suspected when an allergic symptom occurs. The identification unit analyzes the ingredient list image using, for example, AI and identifies an ingredient that may cause an allergy. For example, the identification unit identifies an ingredient causing an allergy based on the ingredient list image of the food consumed by the user. Thereby, the recipe proposal system according to the embodiment can propose a recipe considering the allergy information, the food preferences, the amount of exercise, and the like of the user, and perform accumulation of the special sale information and the ingredient list images, and identification of an allergic ingredient. Specifically, each unit of the present system is implemented as a software module operating on hardware resources including a CPU, a GPU, a memory, and a network interface. The present receiving unit bears a function of normalizing text data and numerical data input via a user interface (UI), converting them into structured data (for example, JSON format), and transmitting them to a subsequent processing unit through an internal bus or an API. The present proposal unit is equipped with a neural network-based recommendation engine, and executes inference processing of performing an inner product operation of an input user profile vector (a floating-point array of dimension N) and a recipe feature vector to calculate a predicted evaluation value. The present acquisition unit includes a Web crawler or an external API linkage module, acquires special sale information from an external server periodically or on demand, performs data cleansing processing (missing value imputation, unification of orthographical variants), and then stores it in a relational database or a NoSQL database. The present accumulation unit links with a distributed file system or object storage, generates a hash value for uploaded image data to perform deduplication, and indexes and saves it together with metadata (shooting date and time, location information, analyzed text). The present identification unit detects a character region in an image using an image recognition AI model (for example, a model having a CNN-based encoder and an RNN-based decoder), outputs a raw material list converted into text by character recognition processing, and further executes a logical operation of pattern-matching the list with an allergen dictionary to identify a corresponding allergen substance.

The recipe proposal system comprises a display unit configured to display an advertisement. The display unit displays an advertisement. The display unit displays an advertisement, for example, at the bottom layer of a recipe page. Since the advertisement is displayed based on an interest or concern of the user, effective monetization is possible. For example, the display unit displays a banner advertisement at the bottom layer of the recipe page being browsed by the user. Further, the display unit can also customize the advertisement based on the interest or concern of the user. For example, the display unit displays a related advertisement based on a past browsing history or a purchase history of the user. Thereby, the recipe proposal system can secure revenue by displaying the advertisement. Part or all of the above-described processing in the display unit may be performed using, for example, AI, or may be performed without using AI. For example, the display unit can select and display an optimal advertisement using an AI model that analyzes the interest or concern of the user. Specifically, the present display unit includes a rendering engine that controls a display device of a user terminal, and draws advertisement content data delivered from a server side as a view component of HTML or a native application. The present display unit utilizes a machine learning model such as a click-through rate (CTR) prediction model for advertisement selection. An input to this CTR prediction model is a multi-dimensional feature vector including a user attribute vector (age, gender, place of residence, etc.), a behavior history vector (past click history, browsing category), and context information (current time, recipe category being browsed). The model adopts an architecture such as logistic regression, Gradient Boosting Decision Tree, or Deep FM (Factorization Machines), and outputs a click probability (a score in a range of 0 to 1) for each advertisement candidate. The present display unit sorts advertisement candidates in descending order based on this output score, selects high-ranked advertisements, and stores them in a display queue. Furthermore, the present display unit has a function of linking with a programmatic advertisement delivery server, conducting a transaction of an advertisement slot in milliseconds through a real-time bidding (RTB) protocol, and dynamically loading and displaying a successful advertisement creative.

The receiving unit can analyze an emotion of the user and adjust an input method of information based on the analyzed emotion of the user. For example, when the user feels stress, the receiving unit provides a simple interface to minimize an input procedure. Further, when the user is relaxed, the receiving unit can provide detailed input options and propose a customizable input method. Furthermore, when the user is in a hurry, the receiving unit can prioritize voice input to enable quick information input. Thereby, by adjusting the input method of information according to the emotion of the user, more appropriate input becomes possible. Estimation of the emotion is realized using an emotion estimation function using, for example, an emotion engine or generative AI. The generative AI is text generation AI (for example, LLM), multimodal generative AI, or the like, but is not limited to such examples. Part or all of the above-described processing in the receiving unit may be performed using, for example, AI, or may be performed without using AI. For example, the receiving unit can input user's facial expression data to the generative AI and cause the generative AI to execute estimation of the emotion. Specifically, the present receiving unit comprises a sensing interface that acquires log data of a camera, a microphone, or a touch panel operation of the user terminal. In emotion estimation processing, the present receiving unit inputs facial image data (for example, an RGB image tensor of 224×224 pixels) acquired from the camera to a convolutional neural network (CNN) to extract facial features (wrinkles between eyebrows, raising of mouth corners, etc.). Further, for voice data acquired from the microphone, the present receiving unit extracts acoustic features such as Mel-Frequency Cepstral Coefficients (MFCC) and inputs them to a Recurrent Neural Network (RNN) or a Transformer model. These models output a probability distribution for each emotion class (“stress”, “relax”, “impatience”, etc.). When a probability of “stress” exceeds a predetermined threshold (for example, 0.7) in the output probability distribution, the present receiving unit sends a command to a UI rendering engine to dynamically rewrite a DOM (Document Object Model) structure of an input form and perform control to switch to a simple mode displaying only essential items. Conversely, when “relax” is dominant, control such as expanding a modal window for detailed settings is performed.

The receiving unit can analyze a past input history of the user and provide an optimal input interface. For example, the receiving unit automatically displays allergy information or food preferences frequently input by the user in the past as candidates. Further, the receiving unit can also preferentially propose an input method (voice, text, etc.) used by the user in the past. Furthermore, the receiving unit can also predict and propose information to be used in a specific time zone from the past input history of the user. Thereby, by analyzing the past input history, an optimal input interface can be provided to the user. Part or all of the above-described processing in the receiving unit may be performed using, for example, AI, or may be performed without using AI. For example, the receiving unit can input past input history data of the user to generative AI and cause the generative AI to execute proposal of an optimal input interface. Specifically, the present receiving unit refers to an operation log database for each user and executes a time-series data analysis algorithm. The present receiving unit implements a sequence prediction model using a recurrent neural network (LSTM or GRU), and receives a history vector of a past input token sequence, input time, and input device type (keyboard, voice, flick input, etc.) as input data. This model outputs a probability distribution of a word or item highly likely to be input next or an input modality to be selected. For example, when the word “chicken” is frequently input in a specific time zone (evening, etc.), the model predicts a high appearance probability of the word. Based on this prediction result, the present receiving unit performs UI control such as dynamically generating and displaying a candidate list of an autocomplete function or highlighting a voice input button. Further, the present receiving unit holds a frequent input pattern in a cache memory and performs edge-side processing of immediately presenting input candidates without going through server communication, thereby reducing latency and improving user experience.

When information is input, the receiving unit can customize an input item based on a current health condition or a lifestyle habit of the user. For example, when the user inputs a result of a health check, the receiving unit adds a necessary nutrient to the input item based on the result. Further, when the user inputs an amount of exercise, the receiving unit can also add a calorie intake amount corresponding to the amount of exercise to the input item. Furthermore, when the user inputs a specific lifestyle habit (for example, vegetarian), the receiving unit can also customize the input item based on the habit. Thereby, by customizing the input item based on the health condition or the lifestyle habit of the user, more appropriate information can be input. Part or all of the above-described processing in the receiving unit may be performed using, for example, AI, or may be performed without using AI. For example, the receiving unit can input health check result data of the user to generative AI and cause the generative AI to execute customization of the input item. Specifically, the present receiving unit links with a healthcare database that manages a health profile (numerical data such as BMI, blood pressure, blood glucose level, cholesterol level, etc.) and lifestyle habit tags (vegan, gluten-free, sugar restriction, etc.) of the user. The present receiving unit uses a rule-based inference engine or a decision tree model to identify a nutrient (iron, vitamin group, saturated fatty acid, etc.) having a risk of being deficient or excessive for the user based on the input health data. For example, when “blood pressure value: 140 mmHg” is given as input data, the inference engine determines that monitoring of “salt intake” is necessary, and issues a command to dynamically add an input field for “salt amount” to an input form generation module. Further, when a large language model (LLM) is used, the present receiving unit inputs text data of the health check result (including an OCR result from a PDF or an image) as a prompt, causes the LLM to output a “recommended management nutrient list” as structured data (JSON, etc.), and executes processing of reconstructing a UI component based on the output.

The receiving unit can estimate an emotion of the user and determine a priority order of information to be input based on the estimated emotion of the user. For example, when the user feels stress, the receiving unit causes important information to be preferentially input and postpones other information. Further, when the user is relaxed, the receiving unit can also cause detailed information to be preferentially input. Furthermore, when the user is in a hurry, the receiving unit can also cause only the most important information to be preferentially input. Thereby, by determining the priority order of information to be input according to the emotion of the user, more appropriate information can be input. Estimation of the emotion is realized using an emotion estimation function using, for example, an emotion engine or generative AI. The generative AI is text generation AI (for example, LLM), multimodal generative AI, or the like, but is not limited to such examples. Part or all of the above-described processing in the receiving unit may be performed using, for example, AI, or may be performed without using AI. For example, the receiving unit can input user's facial expression data to the generative AI and cause the generative AI to execute estimation of the emotion. Specifically, the present receiving unit utilizes a multimodal emotion recognition model to receive user's biological reactions (heart rate variability, skin conductance, etc.) and behavioral features (typing speed, tap pressure, scroll speed) as input vectors, and estimates user's arousal (Arousal) and pleasantness/unpleasantness (Valence) as coordinates on a two-dimensional space. Based on the estimated emotion state, the present receiving unit executes a prioritization algorithm that dynamically assigns a weighting score to each input item (allergy, disliked food, budget, etc.). For example, when it is determined that the arousal is high and the unpleasantness is high (stress state), the present algorithm maximizes weights of essential and safety-related items such as “allergy information” and minimizes weights of additional items such as “detailed taste preference”. Thereafter, the present receiving unit performs sorting processing in the order of item weights, and performs dynamic pagination control such that only the top N items are displayed on the first screen and the remaining items are stored under a “input later” button.

When information is input, the receiving unit can preferentially display a highly relevant input item based on geographical location information of the user. For example, when the user lives in a specific region, the receiving unit preferentially displays ingredients frequently consumed in that region as input items. Further, when the user is traveling, the receiving unit can also preferentially display local specialties or ingredients of a travel destination as input items. Furthermore, when the user uses a supermarket in a specific region, the receiving unit can also preferentially display ingredients sold at that supermarket as input items. Thereby, by preferentially displaying a highly relevant input item in consideration of the geographical location information of the user, more appropriate information can be input. Part or all of the above-described processing in the receiving unit may be performed using, for example, AI, or may be performed without using AI. For example, the receiving unit can input geographical location information data of the user to generative AI and cause the generative AI to execute display of a highly relevant input item. Specifically, the present receiving unit takes latitude and longitude information acquired from GPS, a Wi-Fi access point, or a beacon signal as input, and identifies a country, a prefecture, a municipality, and further a specific commercial facility (supermarket, etc.) by reverse geocoding processing. The present receiving unit issues a spatial search query to a geographic spatial database (GISDB) storing ingredient consumption trends and local specialty data for each region, and acquires an ingredient list highly relevant to the region. Furthermore, the present receiving unit uses a recommendation algorithm such as collaborative filtering to analyze ingredient data frequently input by other users living in the same region, and generates a ranking of input candidates. For example, when location information of “Hokkaido” is obtained, the present system raises scores of “salmon”, “potato”, “dairy products”, etc., and performs UI control to display these ingredients at the top of autocomplete candidates in the input form.

When information is input, the receiving unit can analyze a social media activity of the user and propose a related input item. For example, the receiving unit analyzes a photo of a meal shared by the user on social media and proposes a related ingredient as an input item. Further, the receiving unit can also analyze a recipe of a cooking account followed by the user on social media and propose a related ingredient as an input item. Furthermore, the receiving unit can also analyze a trend of a cooking community in which the user participates on social media and propose a related ingredient as an input item. Thereby, by analyzing the social media activity of the user, a related input item can be proposed. Part or all of the above-described processing in the receiving unit may be performed using, for example, AI, or may be performed without using AI. For example, the receiving unit can input social media activity data of the user to generative AI and cause the generative AI to execute proposal of a related input item. Specifically, the present receiving unit acquires unstructured data such as posted images, posted text, “like” history, and follow lists via an API from a social media account linked with the user's consent. The present receiving unit detects a dish or an ingredient in a posted image using an image recognition model (for example, ResNet or EfficientNet) and outputs an object category (“pasta”, “avocado”, etc.). At the same time, the present receiving unit extracts an ingredient name or a cooking genre from the posted text or a hashtag using a natural language processing model (for example, BERT). These analysis results are aggregated as an interest/concern vector of the user, and presented as a default value or a recommended candidate for “preferred ingredient” or “dish desired to cook” in the input form. For example, when the user frequently reacts to posts related to “camping” or “barbecue” recently, the present receiving unit executes logic to preferentially display input items such as “meat” and “vegetables for skewers”.

The proposal unit can analyze an emotion of the user and adjust a presentation method of the recipe based on the analyzed emotion of the user. For example, when the user feels stress, the proposal unit proposes a simple and easy-to-understand recipe. Further, when the user is relaxed, the proposal unit can also propose a recipe including detailed procedures and arrangement methods. Furthermore, when the user is in a hurry, the proposal unit can also propose a recipe that can be made in a short time. Thereby, by adjusting the presentation method of the recipe according to the emotion of the user, a more appropriate recipe can be proposed. Estimation of the emotion is realized using an emotion estimation function using, for example, an emotion engine or generative AI. The generative AI is text generation AI (for example, LLM), multimodal generative AI, or the like, but is not limited to such examples. Part or all of the above-described processing in the proposal unit may be performed using, for example, AI, or may be performed without using AI. For example, the proposal unit can input emotion data of the user to generative AI and cause the generative AI to execute adjustment of the presentation method of the recipe. Specifically, the present proposal unit is equipped with a natural language generation (NLG) model or a large language model (LLM), and controls a text generation process of recipe data. The present proposal unit takes an emotion label (e.g., “fatigue”, “elation”) and an intensity score output from a preceding emotion estimation module as input, and dynamically changes an instruction (System Prompt) to the LLM using prompt engineering technology. For example, when it is determined that the user is in a “fatigue” state, the present proposal unit gives a constraint condition to the LLM to “summarize the procedure within 3 steps, add words of encouragement, and output in plain language”. Thereby, the LLM summarizes and simplifies original complex recipe data, and outputs text with reduced cognitive load on the user. Conversely, in the case of an “elation” state, an instruction to “add chef's commitment points and scientific explanation” is given to generate detailed and highly entertaining recipe text.

When proposing the recipe, the proposal unit can analyze a past meal history of the user and select an optimal recipe. For example, the proposal unit proposes a similar recipe based on a dish that the user liked to eat in the past. Further, the proposal unit can also propose a recipe that does not include dishes avoided by the user in the past based on the dishes. Furthermore, the proposal unit can also propose a recipe considering nutritional balance from the past meal history of the user. Thereby, by analyzing the past meal history of the user, an optimal recipe can be proposed. Part or all of the above-described processing in the proposal unit may be performed using, for example, AI, or may be performed without using AI. For example, the proposal unit can input past meal history data of the user to generative AI and cause the generative AI to execute selection of an optimal recipe. Specifically, the present proposal unit accumulates user's meal history data (menu name, eating date and time, evaluation score, leftover amount, etc.) as time-series data, and executes a recommendation algorithm such as Matrix Factorization or Neural Collaborative Filtering (NCF). The present proposal unit represents interaction between the user and the recipe as a matrix, and learns a latent factor vector to predict an evaluation value of the user for an uneaten recipe. Furthermore, the present proposal unit uses content-based filtering in combination to search for a new recipe having a vector similar to an ingredient vector or a taste vector (digitized data of sweetness, saltiness, acidity, etc.) of a recipe highly evaluated in the past. Also, from the viewpoint of nutritional balance, the present proposal unit calculates a cumulative value of ingested nutrients for the past week, performs reranking processing of assigning a high weight to a recipe that supplements a deficient nutrient (for example, calcium deficiency), and generates a final recommendation list.

When proposing the recipe, the proposal unit can customize the recipe based on a current health condition or a lifestyle habit of the user. For example, when the user inputs a result of a health check, the proposal unit proposes a recipe including a necessary nutrient based on the result. Further, when the user inputs an amount of exercise, the proposal unit can also propose a recipe considering a calorie intake amount corresponding to the amount of exercise. Furthermore, when the user inputs a specific lifestyle habit (for example, vegetarian), the proposal unit can also propose a recipe based on the habit. Thereby, by customizing the recipe based on the health condition or the lifestyle habit of the user, a more appropriate recipe can be proposed. Part or all of the above-described processing in the proposal unit may be performed using, for example, AI, or may be performed without using AI. For example, the proposal unit can input health check result data of the user to generative AI and cause the generative AI to execute customization of the recipe. Specifically, the present proposal unit comprises an optimization engine that solves a Constraint Satisfaction Problem (CSP) or Linear Programming (LP). The present proposal unit sets a target nutrient range (for example, protein 60 g or more, salt less than 6 g) calculated from user's health data (basal metabolism, activity level, disease risk, etc.) as a constraint condition. Also, the lifestyle habit (vegetarian, etc.) is set as a filtering condition. The present proposal unit applies these conditions to nutritional value data and ingredient attribute data possessed by each recipe in the recipe database, and extracts a recipe set satisfying the conditions. Furthermore, when generative AI is used, the present proposal unit takes a base recipe and a user's health constraint (“low salt for high blood pressure”, etc.) as input, instructs the AI to perform substitution of ingredients (e.g., change normal soy sauce to low-salt soy sauce or dashi) or change of a cooking method (e.g., change deep-frying to grilling), and generates and outputs a customized new recipe procedure and component values.

The proposal unit can estimate an emotion of the user and determine a priority order of recipes based on the estimated emotion of the user. For example, when the user feels stress, the proposal unit preferentially proposes a recipe using an ingredient having a relaxing effect. Further, when the user is relaxed, the proposal unit can also preferentially propose an enjoyable arrangement recipe. Furthermore, when the user is in a hurry, the proposal unit can also preferentially propose a recipe that can be made in a short time. Thereby, by determining the priority order of recipes according to the emotion of the user, a more appropriate recipe can be proposed. Estimation of the emotion is realized using an emotion estimation function using, for example, an emotion engine or generative AI. The generative AI is text generation AI (for example, LLM), multimodal generative AI, or the like, but is not limited to such examples. Part or all of the above-described processing in the proposal unit may be performed using, for example, AI, or may be performed without using AI. For example, the proposal unit can input emotion data of the user to generative AI and cause the generative AI to execute determination of the priority order of recipes. Specifically, the present proposal unit uses a ranking model (Learning to Rank) that has learned a correlation between an emotion state and a recipe attribute (cooking time, difficulty level, efficacy of ingredient, taste tendency). As input, an emotion vector of the user (e.g., [stress: 0.8, fatigue: 0.6]) and a feature vector of the recipe are received. The model outputs a score indicating how much each recipe is accepted in the current emotion state of the user. For example, when a stress value is high, a high score is assigned to a recipe using an ingredient containing GABA or tryptophan (banana, dairy products, etc.) or a recipe with few steps and low failure risk. The present proposal unit sorts a recipe list based on this score and presents high-ranked ones to the user. Also, using reinforcement learning, the model is continuously updated using a user's reaction (click, cooking completion, evaluation) to the proposed recipe as a reward to improve matching accuracy between the emotion and the recipe.

When proposing the recipe, the proposal unit can preferentially propose a highly relevant recipe based on geographical location information of the user. For example, when the user lives in a specific region, the proposal unit preferentially proposes a recipe using a special product of that region. Further, when the user is traveling, the proposal unit can also preferentially propose a recipe using a special product of a travel destination. Furthermore, when the user uses a supermarket in a specific region, the proposal unit can also preferentially propose a recipe using ingredients sold at that supermarket. Thereby, by preferentially proposing a highly relevant recipe in consideration of the geographical location information of the user, a more appropriate recipe can be proposed. Part or all of the above-described processing in the proposal unit may be performed using, for example, AI, or may be performed without using AI. For example, the proposal unit can input geographical location information data of the user to generative AI and cause the generative AI to execute proposal of a highly relevant recipe. Specifically, the present proposal unit identifies an area (prefecture, tourist spot, trade area) where the user is currently staying using geofencing technology based on location information (latitude/longitude) transmitted from the user terminal. The present proposal unit refers to a “seasonal ingredient database” or a “local cuisine database” for each region, and acquires ingredients or cooking tags associated with the identified area. Next, the present proposal unit adds these tags (e.g., “Kyoto”, “Kyoto vegetables”, “boiled tofu”) as query parameters of a recipe search engine, and performs weighting processing to boost a search score. Furthermore, by linking with an inventory API of a nearby supermarket, the present proposal unit preferentially filters and proposes a recipe using an ingredient that is in stock at a store within a radius of N km from the current location and is on special sale, thereby maximizing user convenience and economic merit.

When proposing the recipe, the proposal unit can analyze a social media activity of the user and propose a related recipe. For example, the proposal unit analyzes a photo of a meal shared by the user on social media and proposes a related recipe. Further, the proposal unit can also analyze a recipe of a cooking account followed by the user on social media and propose a related recipe. Furthermore, the proposal unit can also analyze a trend of a cooking community in which the user participates on social media and propose a related recipe. Thereby, by analyzing the social media activity of the user, a related recipe can be proposed. Part or all of the above-described processing in the proposal unit may be performed using, for example, AI, or may be performed without using AI. For example, the proposal unit can input social media activity data of the user to generative AI and cause the generative AI to execute proposal of a related recipe. Specifically, the present proposal unit uses a Graph Neural Network (GNN) to construct and analyze a social graph having users, posts, follow relationships, recipes, ingredients, etc. as nodes and their relationships as edges. The present proposal unit searches for a recipe node located in the vicinity of (strongly relevant to) a user node, and identifies a recipe in which the user is highly likely to be interested by a link prediction algorithm. For example, when an influencer followed by the user posts a specific recipe, an edge weight to that recipe node increases, and a proposal score rises. Also, in trend analysis, the present proposal unit extracts a buzzword (popular dish name or ingredient) from a large number of posts on SNS using natural language processing, and performs processing of picking up a recipe including the trend element in real time and inserting it into a proposal list.

The acquisition unit can analyze an emotion of the user and adjust an acquisition timing of the special sale information based on the analyzed emotion of the user. For example, when the user feels stress, the acquisition unit acquires special sale information early and makes a proposal for reducing stress. Further, when the user is relaxed, the acquisition unit can also acquire special sale information at a normal timing and make a proposal in a relaxed state. Furthermore, when the user is in a hurry, the acquisition unit can also acquire special sale information quickly and make it available immediately. Thereby, by adjusting the acquisition timing of the special sale information according to the emotion of the user, more appropriate special sale information can be acquired. Estimation of the emotion is realized using an emotion estimation function using, for example, an emotion engine or generative AI. The generative AI is text generation AI (for example, LLM), multimodal generative AI, or the like, but is not limited to such examples. Part or all of the above-described processing in the acquisition unit may be performed using, for example, AI, or may be performed without using AI. For example, the acquisition unit can input emotion data of the user to generative AI and cause the generative AI to execute adjustment of the acquisition timing of the special sale information. Specifically, the present acquisition unit implements a task scheduling algorithm, and dynamically controls a data acquisition request (polling interval or priority) to an external server. The present acquisition unit changes a priority order of a request queue based on an input (emotion state: impatience, stress, etc.) from an emotion estimation module. For example, when it is determined that the user is in an “impatience” state (in a hurry), the present acquisition unit issues a synchronous request (Force Sync) immediately without waiting for a periodic update performed in the background, and acquires and analyzes special sale information in a top-priority thread. Also, when the user feels “stress”, the present acquisition unit sets a scheduler to perform push notification at a timing earlier than usual (for example, one hour before return time), and operates to provide a psychological reward by profitable information at an early stage. For this control, reinforcement learning (Contextual Bandits) using a user's reaction rate as a reward is applied to learn an optimal timing strategy.

The acquisition unit can analyze past special sale information of the nearby supermarket and select an optimal acquisition method. For example, the acquisition unit analyzes a frequency or a pattern of special sales based on the past special sale information of the nearby supermarket, and selects an optimal acquisition timing. Further, the acquisition unit can also analyze a tendency of special sales based on the past special sale information of the nearby supermarket, and select an optimal acquisition method. Furthermore, the acquisition unit can also analyze a type of special sales based on the past special sale information of the nearby supermarket, and select an optimal acquisition method. Thereby, by analyzing the past special sale information, an optimal acquisition method can be selected. Part or all of the above-described processing in the acquisition unit may be performed using, for example, AI, or may be performed without using AI. For example, the acquisition unit can input past special sale information data of the nearby supermarket to generative AI and cause the generative AI to execute selection of an optimal acquisition method. Specifically, the present acquisition unit uses a time-series data prediction model (for example, ARIMA, Prophet, or LSTM) to learn a special sale pattern (periodicity of day of week, time zone, product category) of each supermarket. Input data is log data of flyer update dates and times and special sale start times for the past several months. The model predicts and outputs a time frame (Time Window) in which a probability that the next special sale information is updated is high. The present acquisition unit sets a schedule of a crawler or an API request in accordance with this predicted time frame, thereby maximizing freshness of information while reducing useless traffic. Also, the present acquisition unit learns a tendency of site structure change or access restriction, and automatically adjusts a scraping frequency or the number of parallels, thereby executing an optimal data acquisition strategy considering a server load.

When acquiring the special sale information, the acquisition unit can perform filtering based on a current purchase history or a field of interest of the user. For example, the acquisition unit preferentially acquires special sale information of high interest based on the purchase history of the user. Further, the acquisition unit can also preferentially acquire related special sale information based on the field of interest of the user. Furthermore, the acquisition unit can also combine the purchase history and the field of interest of the user to filter and acquire optimal special sale information. Thereby, by performing filtering based on the purchase history or the field of interest of the user, more appropriate special sale information can be acquired. Part or all of the above-described processing in the acquisition unit may be performed using, for example, AI, or may be performed without using AI. For example, the acquisition unit can input purchase history data of the user to generative AI and cause the generative AI to execute filtering of the special sale information. Specifically, the present acquisition unit comprises a filtering engine that performs similarity calculation (cosine similarity, etc.) between a purchase history vector of the user (frequency distribution of product categories purchased in the past) and a special sale product vector (feature quantities such as product category, discount rate, production area, etc.). The present acquisition unit applies threshold processing based on this similarity score to a huge special sale information list (Raw Data) acquired from an external source, and excludes information having low relevance to the user (for example, pet food special sale information for a user who does not keep a pet) at an initial stage. Also, using an AI model (for example, a Gradient Boosting Tree such as LightGBM), the present acquisition unit predicts a “purchase probability” of the user for each special sale product, sorts data in descending order of probability, and passes it to a subsequent processing unit, thereby improving throughput of the entire system and user satisfaction.

The acquisition unit can analyze an emotion of the user and determine a priority order of the special sale information to be acquired based on the analyzed emotion of the user. For example, when the user feels stress, the acquisition unit preferentially acquires special sale information useful for stress reduction. Further, the acquisition unit can also preferentially acquire special sale information having a relaxing effect when the user is relaxed. Furthermore, when the user is in a hurry, the acquisition unit can also preferentially acquire special sale information that can be used immediately. Thereby, by determining the priority order of the special sale information to be acquired according to the emotion of the user, more appropriate special sale information can be acquired. Estimation of the emotion is realized using an emotion estimation function using, for example, an emotion engine or generative AI. The generative AI is text generation AI (for example, LLM), multimodal generative AI, or the like, but is not limited to such examples. Part or all of the above-described processing in the acquisition unit may be performed using, for example, AI, or may be performed without using AI. For example, the acquisition unit can input emotion data of the user to generative AI and cause the generative AI to execute determination of the priority order of the special sale information. Specifically, the present acquisition unit performs metadata tagging processing according to an emotion context. The present acquisition unit takes an emotion estimation result (e.g., stress) as input, and sets an acquisition priority flag for a special sale item having a “stress relief” tag (e.g., a tag associated with chocolate, herbal tea, bath salts, etc.) in a product database. Also, when the user is “in a hurry”, the present acquisition unit executes a dynamic crawling strategy of preferentially crawling special sale information of prepared dishes or instant foods requiring no cooking and postponing information of fresh foods. This prioritization is controlled by a multi-objective optimization algorithm, and is determined by calculating a balance between an emotional need of the user and an economic merit (discount rate).

The acquisition unit can preferentially acquire highly relevant information in consideration of geographical location information of the user when acquiring the special sale information. For example, when the user lives in a specific area, the acquisition unit preferentially acquires special sale information of a supermarket in that area. Also, when the user is traveling, the acquisition unit can preferentially acquire special sale information of a supermarket at a travel destination. Furthermore, when the user uses a supermarket in a specific area, the acquisition unit can preferentially acquire special sale information of that supermarket. Thereby, by preferentially acquiring highly relevant information in consideration of the geographical location information of the user, more appropriate special sale information can be acquired. Part or all of the above-described processing in the acquisition unit may be performed using, for example, AI, or may be performed without using AI. For example, the acquisition unit can input geographical location information data of the user to a generative AI and cause the generative AI to execute acquisition of highly relevant special sale information. Specifically, the present acquisition unit cooperates with a location-based service (LBS) to generate a list of stores within a radius R meters centered on the current location coordinates of the user in real time. The present acquisition unit issues a query to a special sale information database or an external API using a store ID included in this store list as a key. At this time, predictive caching is performed in which a movement velocity vector of the user is analyzed and information on a store in a movement direction is acquired in advance. For example, when the user is moving toward a station, special sale information of supermarkets and convenience stores around the station is preferentially put into an acquisition queue. In addition, when it is determined that the user is traveling (when the user is away from a residence by a certain distance or more), an acquisition process for a normally used store is temporarily stopped, and an acquisition process for store information around the current location is activated, thereby efficiently allocating resources.

The acquisition unit can analyze a social media activity of the user and acquire related information when acquiring the special sale information. For example, the acquisition unit acquires related special sale information based on special sale information shared by the user on social media. Also, the acquisition unit can acquire related special sale information based on special sale information of a supermarket followed by the user on social media. Furthermore, the acquisition unit can analyze a trend of a special sale community in which the user participates on social media and acquire related special sale information. Thereby, by analyzing the social media activity of the user, related special sale information can be acquired. Part or all of the above-described processing in the acquisition unit may be performed using, for example, AI, or may be performed without using AI. For example, the acquisition unit can input social media activity data of the user to a generative AI and cause the generative AI to execute acquisition of related special sale information. Specifically, the present acquisition unit has a social listening function, and monitors and collects posts including specific hashtags (e.g., #special sale, #super cheap) and keywords in real time. The present acquisition unit uses a natural language processing (NLP) model to perform entity extraction (NER) of a store name, a product name, and price information from posts of the user themselves or accounts being followed. The extracted information is converted into structured data and integrated into a special sale database of the system. Furthermore, the present acquisition unit uses a community detection algorithm on a social graph to identify special sale information that is a topic within a cluster to which the user belongs, and uses the information as a trigger to acquire support from an official information source (flyer, etc.). This makes it possible to quickly capture word-of-mouth-based special sale information before official information is released, region-limited guerrilla sale information, and the like.

The accumulation unit can analyze an emotion of the user and adjust an accumulation method of the ingredient list images based on the analyzed emotion of the user. For example, when the user feels stress, the accumulation unit accumulates the ingredient list images with a simple interface. Also, when the user is relaxed, the accumulation unit can accumulate ingredient list images including detailed information. Furthermore, when the user is in a hurry, the accumulation unit can allow the ingredient list images to be accumulated quickly. Thereby, by adjusting the accumulation method of the ingredient list images according to the emotion of the user, more appropriate ingredient list images can be accumulated. The estimation of the emotion is realized using an emotion estimation function using, for example, an emotion engine or a generative AI. The generative AI is a text generative AI (e.g., LLM), a multimodal generative AI, or the like, but is not limited to such examples. Part or all of the above-described processing in the accumulation unit may be performed using, for example, AI, or may be performed without using AI. For example, the accumulation unit can input emotion data of the user to a generative AI and cause the generative AI to execute adjustment of the accumulation method of the ingredient list images. Specifically, the present accumulation unit has a state machine that dynamically controls a workflow of image upload processing. When an emotion estimation result is “impatience (in a hurry),” the present accumulation unit shifts to a “quick save mode” in which a compression rate of image data is increased to shorten an upload time, and OCR analysis and metadata addition processing on a server side are executed asynchronously (in the background). In this mode, a completion notification to the user is performed fastest. On the other hand, when it is determined that the user is “relaxed” and is “detail-oriented,” the present accumulation unit provides an “interactive save mode” in which an image is saved with high resolution as it is, and an OCR analysis result is displayed on the spot to request correction from the user. This control optimizes a trade-off between data quality and accumulation speed while minimizing a psychological burden on the user.

The accumulation unit can analyze a past consumption history of the user and select an optimal accumulation method when accumulating the ingredient list images. For example, the accumulation unit selects an optimal accumulation method based on ingredient list images of food consumed by the user in the past. Also, the accumulation unit can preferentially accumulate ingredient list images of food frequently consumed from the past consumption history of the user. Furthermore, the accumulation unit can analyze the past consumption history of the user and accumulate ingredient list images in consideration of a nutritional balance. Thereby, by analyzing the past consumption history of the user, an optimal accumulation method of the ingredient list images can be selected. Part or all of the above-described processing in the accumulation unit may be performed using, for example, AI, or may be performed without using AI. For example, the accumulation unit can input past consumption history data of the user to a generative AI and cause the generative AI to execute selection of an optimal accumulation method. Specifically, the present accumulation unit uses an image hashing technology (Perceptual Hashing) and a feature point matching algorithm to determine whether an image to be newly uploaded is identical or similar to an image accumulated in the past or a known product image in a database. When the identical image exists, the present accumulation unit executes a deduplication process of saving only a reference link (pointer) to existing data without redundantly saving actual data, thereby saving storage capacity. In addition, for frequently consumed food, an image is held in a local cache to improve display speed. Furthermore, based on a nutritional balance analysis, processing of automatically raising a data preservation level, such as assigning a flag as “important data” to an image of food containing a lacking nutrient and performing redundant backup, is performed.

The accumulation unit can customize an accumulation item based on a current health condition or a lifestyle habit of the user when accumulating the ingredient list images. For example, when the user inputs a result of a health checkup, the accumulation unit accumulates ingredient list images containing necessary nutrients based on the result. Also, when the user inputs an amount of exercise, the accumulation unit can accumulate ingredient list images in consideration of a calorie intake corresponding to the amount of exercise. Furthermore, when the user inputs a specific lifestyle habit (e.g., vegetarian), the accumulation unit can accumulate ingredient list images based on the habit. Thereby, by customizing the accumulation item based on the health condition or the lifestyle habit of the user, more appropriate ingredient list images can be accumulated. Part or all of the above-described processing in the accumulation unit may be performed using, for example, AI, or may be performed without using AI. For example, the accumulation unit can input health checkup result data of the user to a generative AI and cause the generative AI to execute customization of the accumulation item. Specifically, the present accumulation unit performs semantic tagging based on a health profile of the user on text data (raw materials, nutritional component display) extracted from an image. For example, when a user with high blood pressure saves an image, the present accumulation unit automatically extracts a numerical value of “salt equivalent” from an OCR result, adds the value to the image as metadata, and adds the value to a search index of a database. In the case of a vegetarian user, the presence or absence of animal-derived ingredients (gelatin, milk components, etc.) included in a raw material list is analyzed, and a flag of “vegetarian compatible/incompatible” is automatically assigned and accumulated. This realizes accumulation as structured data directly linked to health management of the user, rather than mere storage of images.

The accumulation unit can analyze an emotion of the user and determine a priority order of the ingredient list images to be accumulated based on the analyzed emotion of the user. For example, when the user feels stress, the accumulation unit preferentially accumulates ingredient list images useful for stress reduction. Also, when the user is relaxed, the accumulation unit can preferentially accumulate ingredient list images having a relaxing effect. Furthermore, when the user is in a hurry, the accumulation unit can preferentially accumulate ingredient list images that can be used immediately. Thereby, by determining the priority order of the ingredient list images to be accumulated according to the emotion of the user, more appropriate ingredient list images can be accumulated. The estimation of the emotion is realized using an emotion estimation function using, for example, an emotion engine or a generative AI. The generative AI is a text generative AI (e.g., LLM), a multimodal generative AI, or the like, but is not limited to such examples. Part or all of the above-described processing in the accumulation unit may be performed using, for example, AI, or may be performed without using AI. For example, the accumulation unit can input emotion data of the user to a generative AI and cause the generative AI to execute determination of the priority order of the ingredient list images. Specifically, the present accumulation unit adopts a priority queuing mechanism that rearranges a processing order based on an emotion context when a plurality of image upload requests stay in a queue. When the user is in a “stress” state, the present accumulation unit identifies images of food containing a large amount of ingredients related to stress reduction (e.g., GABA, vitamin C, calcium) by image analysis, and sets processing priorities thereof to the highest level. This speeds up a response when the user confirms the food information in an application immediately after or keeps an intake record. Also, even when a server load is high, preservation of data matching a current emotion state of the user is prioritized, and processing of other data is delayed, thereby optimizing system resource allocation.

The accumulation unit can preferentially accumulate a highly relevant image in consideration of geographical location information of the user when accumulating the ingredient list images. For example, when the user lives in a specific area, the accumulation unit preferentially accumulates ingredient list images of food frequently consumed in that area. Also, when the user is traveling, the accumulation unit can preferentially accumulate ingredient list images of special products at a travel destination. Furthermore, when the user uses a supermarket in a specific area, the accumulation unit can preferentially accumulate ingredient list images of food sold at that supermarket. Thereby, by preferentially accumulating a highly relevant image in consideration of the geographical location information of the user, more appropriate ingredient list images can be accumulated. Part or all of the above-described processing in the accumulation unit may be performed using, for example, AI, or may be performed without using AI. For example, the accumulation unit can input geographical location information data of the user to a generative AI and cause the generative AI to execute accumulation of highly relevant ingredient list images. Specifically, the present accumulation unit adopts a distributed database system (e.g., Cassandra or DynamoDB), and distributes and arranges data using geographical location information (Geohash) as a sharding key. When the user uploads an image in a specific area (e.g., Hokkaido), the present accumulation unit preferentially writes data to a data center or an edge server in that area to minimize access latency. In addition, a “common food” model for each region is learned, and when there is a high probability that an uploaded image is a special product of that region, detailed analysis processing (production area identification, brand identification) is automatically triggered, and region-specific metadata is added and accumulated. This autonomously improves the accuracy of a food database for each region.

The accumulation unit can analyze a social media activity of the user and accumulate a related image when accumulating the ingredient list images. For example, the accumulation unit accumulates related ingredient list images based on ingredient list images of food shared by the user on social media. Also, the accumulation unit can accumulate related ingredient list images based on ingredient list images of a food account followed by the user on social media. Furthermore, the accumulation unit can analyze a trend of a food community in which the user participates on social media and accumulate related ingredient list images. Thereby, by analyzing the social media activity of the user, related ingredient list images can be accumulated. Part or all of the above-described processing in the accumulation unit may be performed using, for example, AI, or may be performed without using AI. For example, the accumulation unit can input social media activity data of the user to a generative AI and cause the generative AI to execute accumulation of related ingredient list images. Specifically, the present accumulation unit has a function of crawling image data on social media and automatically collecting and accumulating food images related to the user. The present accumulation unit uses an image recognition AI to filter and extract images in which an “ingredient list” or a “package back surface” is captured from images on an SNS. For the extracted image, the present accumulation unit automatically determines whether to link with an ID of the user and save the image in a private storage or to anonymize and save the image as a part of a public food database based on privacy settings of the user and copyright information of the image (rights notation in metadata, etc.). In addition, when ingredient list images of specific food (e.g., trendy imported confectionery) are rapidly increasing on the SNS by trend analysis, the images are preferentially taken into a master database of the system so that all users can refer to them.

The identification unit can analyze an emotion of the user and adjust an identification method of an allergic ingredient based on the analyzed emotion of the user. For example, when the user feels stress, the identification unit provides a simple and easy-to-understand identification method. Also, when the user is relaxed, the identification unit can provide an identification method including detailed information. Furthermore, when the user is in a hurry, the identification unit can provide a method capable of quickly identifying an allergic ingredient. Thereby, by adjusting the identification method of the allergic ingredient according to the emotion of the user, a more appropriate allergic ingredient can be identified. The estimation of the emotion is realized using an emotion estimation function using, for example, an emotion engine or a generative AI. The generative AI is a text generative AI (e.g., LLM), a multimodal generative AI, or the like, but is not limited to such examples. Part or all of the above-described processing in the identification unit may be performed using, for example, AI, or may be performed without using AI. For example, the identification unit can input emotion data of the user to a generative AI and cause the generative AI to execute adjustment of the identification method of the allergic ingredient. Specifically, the present identification unit dynamically changes a balance between sensitivity and specificity of allergy determination logic and a granularity of result presentation. When the user feels “anxiety” or “stress,” the present identification unit adopts determination logic tilted to a “safe side,” issues a warning over a wide range including a possibility of minute contamination (contamination), and displays a result with intuitive icons and short text such as “contains milk ingredients” or “danger” instead of complex chemical substance names on a UI. On the other hand, when the user is “relaxed” and has a high willingness to learn, a report including detailed scientific data such as content of ingredients, origin, and risk of cross-reactivity is generated and displayed. This adjustment is executed by a rule-based control module that receives an emotion label as an input.

The identification unit can analyze a past allergy history of the user and select an optimal identification method when identifying the allergic ingredient. For example, the identification unit selects an optimal identification method based on the past allergy history of the user. Also, the identification unit can preferentially identify an ingredient that frequently showed an allergic reaction from the past allergy history of the user. Furthermore, the identification unit can analyze the past allergy history of the user and identify a pattern of the allergic reaction. Thereby, by analyzing the past allergy history of the user, an optimal identification method of the allergic ingredient can be selected. Part or all of the above-described processing in the identification unit may be performed using, for example, AI, or may be performed without using AI. For example, the identification unit can input past allergy history data of the user to a generative AI and cause the generative AI to execute selection of an optimal identification method. Specifically, the present identification unit calculates an allergen risk for each user using a Bayesian estimation model or a probabilistic graphical model. Input data is a past onset history (date and time, consumed food, severity of symptoms) and a list of ingredients contained in the food. The model calculates a posterior probability that each ingredient is a cause of an allergic reaction. The present identification unit applies a “focused identification mode” in which a detection threshold during image analysis is lowered so that even minute characters can be detected for an ingredient having a high probability (e.g., “buckwheat” or “peanut”), or extensive dictionary matching including synonyms (e.g., “groundnut”, “Nanjing bean”) is performed. This realizes highly accurate identification that does not overlook a risk factor specific to the user.

The identification unit can customize an identification item based on a current health condition or a lifestyle habit of the user when identifying the allergic ingredient. For example, when the user inputs a result of a health checkup, the identification unit identifies an allergic ingredient based on the result. Also, when the user inputs an amount of exercise, the identification unit can identify an allergic ingredient corresponding to the amount of exercise. Furthermore, when the user inputs a specific lifestyle habit (e.g., vegetarian), the identification unit can identify an allergic ingredient based on the habit. Thereby, by customizing the identification item based on the health condition or the lifestyle habit of the user, a more appropriate allergic ingredient can be identified. Part or all of the above-described processing in the identification unit may be performed using, for example, AI, or may be performed without using AI. For example, the identification unit can input health checkup result data of the user to a generative AI and cause the generative AI to execute customization of the identification item. Specifically, the present identification unit refers to a cross-reference database based on medical knowledge. For example, when the user inputs health information of “hay fever (birch pollen),” the present identification unit adds fruits (apple, peach, etc.) that may cause oral allergy syndrome (OAS) to the identification item as “ingredients requiring caution” (consideration of cross-reactivity). Also, when the user selects a lifestyle habit of “gluten-free,” the present identification unit adds not only wheat but also barley, rye, and hybrids thereof to a detection target list, and extends identification logic so as to detect hidden gluten (soy sauce, malt extract, etc.). In this way, the present identification unit executes ingredient screening adapted to biological characteristics and lifestyle of the user beyond mere allergen identification.

The identification unit can analyze an emotion of the user and determine a priority order of the allergic ingredient to be identified based on the analyzed emotion of the user. For example, when the user feels stress, the identification unit preferentially identifies an allergic ingredient useful for stress reduction. Also, when the user is relaxed, the identification unit can preferentially identify an allergic ingredient having a relaxing effect. Furthermore, when the user is in a hurry, the identification unit can preferentially identify an allergic ingredient that can be used immediately. Thereby, by determining the priority order of the allergic ingredient to be identified according to the emotion of the user, a more appropriate allergic ingredient can be identified. The estimation of the emotion is realized using an emotion estimation function using, for example, an emotion engine or a generative AI. The generative AI is a text generative AI (e.g., LLM), a multimodal generative AI, or the like, but is not limited to such examples. Part or all of the above-described processing in the identification unit may be performed using, for example, AI, or may be performed without using AI. For example, the identification unit can input emotion data of the user to a generative AI and cause the generative AI to execute determination of the priority order of the allergic ingredient. Specifically, the present identification unit changes a sorting algorithm when displaying a list of a plurality of detected ingredients according to an emotion context. Normally, the ingredients are displayed in order of risk of allergens, but when the user is “in a hurry,” the present identification unit displays “ingredients that may cause severe anaphylaxis shock (specified raw materials 7 items, etc.)” requiring immediate judgment at the top, and hides or collapses other minor ingredients or recommended ingredients. Also, when the user feels “anxiety,” the present identification unit preferentially checks not only allergens but also presence or absence of additives (preservatives, coloring agents) that may incite anxiety, and executes logic to highlight a result thereof (relief material such as “no preservatives used”).

The identification unit can preferentially identify a highly relevant ingredient in consideration of geographical location information of the user when identifying the allergic ingredient. For example, when the user lives in a specific area, the identification unit preferentially identifies an allergic ingredient of food frequently consumed in that area. Also, when the user is traveling, the identification unit can preferentially identify an allergic ingredient of a special product at a travel destination. Furthermore, when the user uses a supermarket in a specific area, the identification unit can preferentially identify an allergic ingredient of food sold at that supermarket. Thereby, by preferentially identifying a highly relevant ingredient in consideration of the geographical location information of the user, a more appropriate allergic ingredient can be identified. Part or all of the above-described processing in the identification unit may be performed using, for example, AI, or may be performed without using AI. For example, the identification unit can input geographical location information data of the user to a generative AI and cause the generative AI to execute identification of a highly relevant allergic ingredient. Specifically, the present identification unit has a localization function corresponding to a food labeling standard and a language difference for each region. When the user is traveling abroad (determined by GPS information), the present identification unit automatically loads an OCR model of a language of that country (e.g., Thai or French) and analyzes a local ingredient display. Furthermore, ingredient identification is performed based on a risk standard different from that in Japan by referring to a database regarding allergens specific to that region (e.g., specific spices and insect food in Asian regions, frequency of use of peanut oil in Western countries). Also, even in Japan, for region-limited processed food, collation with a local product database is performed, and ingredient information not listed in a general database is supplemented to improve identification accuracy.

The identification unit can analyze a social media activity of the user and identify a related ingredient when identifying the allergic ingredient. For example, the identification unit identifies a related ingredient based on an allergic ingredient of food shared by the user on social media. Also, the identification unit can identify a related ingredient based on an allergic ingredient of a food account followed by the user on social media. Furthermore, the identification unit can analyze a trend of a food community in which the user participates on social media and identify a related allergic ingredient. Thereby, by analyzing the social media activity of the user, a related allergic ingredient can be identified. Part or all of the above-described processing in the identification unit may be performed using, for example, AI, or may be performed without using AI. For example, the identification unit can input social media activity data of the user to a generative AI and cause the generative AI to execute identification of a related allergic ingredient. Specifically, the present identification unit performs ingredient identification utilizing collective intelligence (Crowd Sourcing) on social media. The present identification unit searches whether an image similar to a food image taken by the user is posted on an SNS, and if it exists, analyzes text accompanying the post (comments such as “I got hives after eating this” or “Be careful because it contains eggs”) by natural language processing. Thereby, risk information regarding ingredients not described in an official ingredient list or having ambiguous descriptions (“secret ingredient” or shared information of a production line) is extracted and added to an identification result as “reference information derived from SNS.” Also, a “new allergen” or “ingredient change information” that is a topic in a specific food community is detected as a trend, and a dictionary of an identification engine is updated in real time.

The display unit can analyze an emotion of the user and adjust a display method of the advertisement based on the analyzed emotion of the user. For example, when the user feels stress, the display unit displays a simple and highly visible advertisement. Also, when the user is relaxed, the display unit can display an advertisement including detailed information. Furthermore, when the user is in a hurry, the display unit can display an advertisement focusing on main points. Thereby, by adjusting the display method of the advertisement according to the emotion of the user, more effective advertisement display becomes possible. The estimation of the emotion is realized using an emotion estimation function using, for example, an emotion engine or a generative AI. The generative AI is a text generative AI (e.g., LLM), a multimodal generative AI, or the like, but is not limited to such examples. Part or all of the above-described processing in the display unit may be performed using, for example, AI, or may be performed without using AI. For example, the display unit can input emotion data of the user to a generative AI and cause the generative AI to execute adjustment of the display method of the advertisement. Specifically, the present display unit applies dynamic creative optimization (DCO) technology to generate and change a layout, a color scheme, and a text amount of an advertisement in real time. When an emotion estimation result is “stress,” the present display unit selects a still image banner excluding animation and blinking in order to reduce a cognitive load, and applies a calm color scheme template of cold colors. Conversely, in a state of “relaxed” or “bored,” an interactive video advertisement or a carousel type advertisement is displayed to increase engagement of the user. Also, using the generative AI, processing of rewriting a catchphrase of the advertisement according to the emotion of the user (e.g., “a moment of healing” for a tired person, “let's be active energetically” for a healthy person) is also executed.

The display unit can analyze a past browsing history of the user and select the advertisement when displaying the advertisement. For example, the display unit displays an advertisement related to a product or service browsed by the user in the past. Also, the display unit can select an advertisement in which the user is likely to be interested from the past browsing history of the user. Furthermore, the display unit can analyze the past browsing history of the user and select a most effective advertisement. Thereby, by analyzing the past browsing history of the user, an optimal advertisement can be selected. Part or all of the above-described processing in the display unit may be performed using, for example, AI, or may be performed without using AI. For example, the display unit can input past browsing history data of the user to a generative AI and cause the generative AI to execute selection of an optimal advertisement. Specifically, the present display unit cooperates with a DSP (Demand-Side Platform) module that executes a retargeting (remarketing) algorithm. The present display unit analyzes a browsing history (page URL, stay time, scroll depth) of the user in association with a cookie or an advertisement ID (IDFA/AAID), and estimates a position on a purchase funnel (cognition, interest, consideration, immediately before purchase) of the user. For example, for a user who has browsed a recipe page of a specific cooking utensil many times but has not purchased it, an advertisement with a discount coupon for the cooking utensil is displayed. A Multi-Armed Bandit algorithm is used for this selection process, and an advertisement that maximizes a click-through rate (CTR) or a conversion rate (CVR) is automatically selected while balancing exploration (test of a new advertisement) and exploitation (display of a highly effective advertisement).

The display unit can customize the advertisement based on a current interest or concern of the user when displaying the advertisement. For example, the display unit displays an advertisement related to a product or service in which the user is currently interested. Also, the display unit can customize a related advertisement based on the current concern of the user. Furthermore, the display unit can analyze the current interest or concern of the user and customize a most effective advertisement. Thereby, by customizing the advertisement based on the current interest or concern of the user, more effective advertisement display becomes possible. Part or all of the above-described processing in the display unit may be performed using, for example, AI, or may be performed without using AI. For example, the display unit can input current interest or concern data of the user to a generative AI and cause the generative AI to execute customization of the advertisement. Specifically, the present display unit performs real-time semantic analysis obtained by evolving context targeting technology. The present display unit inputs text content (ingredients, cooking method, sense of season) of a recipe page currently browsed by the user and a search keyword input immediately before by the user to a natural language processing model (BERT, etc.) to generate a current “interest vector.” An inner product of this vector and a “product vector” possessed by advertisement inventory is calculated, and an advertisement with the highest similarity is selected in milliseconds. Furthermore, using the generative AI, advanced customization such as synthesizing a completed photo of the recipe currently being browsed into an image of the selected advertisement or dynamically inserting a message related to the recipe (e.g., “this wine goes well with this dish”) is performed, thereby dramatically improving relevance and appeal of the advertisement.

The display unit can analyze an emotion of the user and determine a priority order of the advertisement based on the analyzed emotion of the user. For example, when the user feels stress, the display unit preferentially displays an advertisement useful for stress reduction. Also, when the user is relaxed, the display unit can preferentially display an advertisement having a relaxing effect. Furthermore, when the user is in a hurry, the display unit can preferentially display an advertisement that can be used immediately. Thereby, by determining the priority order of the advertisement according to the emotion of the user, more effective advertisement display becomes possible. The estimation of the emotion is realized using an emotion estimation function using, for example, an emotion engine or a generative AI. The generative AI is a text generative AI (e.g., LLM), a multimodal generative AI, or the like, but is not limited to such examples. Part or all of the above-described processing in the display unit may be performed using, for example, AI, or may be performed without using AI. For example, the display unit can input emotion data of the user to a generative AI and cause the generative AI to execute determination of the priority order of the advertisement. Specifically, the present display unit has bidding logic (Bid Modifiers) centered on an emotion state. An advertiser can set a bid unit price in advance for an emotion segment such as “user feeling stress” or “happy user.” The present display unit recalculates an effective bid amount (eCPM) of each advertisement campaign based on the estimated emotion and determines a display rank. For example, when the user is in a “stress” state, a coefficient is multiplied by a bid amount of an advertisement for a relaxation service or a sweet product to raise it, and the advertisement is preferentially displayed. Also, when the user is “in a hurry,” control is performed such that a priority of a video advertisement for brand awareness is lowered and a priority of a banner advertisement for a convenience store product that can be purchased immediately is raised.

The display unit can preferentially display a highly relevant advertisement in consideration of geographical location information of the user when displaying the advertisement. For example, when the user lives in a specific area, the display unit preferentially displays an advertisement for a product or service provided in that area. Also, when the user is traveling, the display unit can preferentially display an advertisement for a product or service provided at a travel destination. Furthermore, when the user uses a supermarket in a specific area, the display unit can preferentially display an advertisement for a product or service provided at that supermarket. Thereby, by preferentially displaying a highly relevant advertisement in consideration of the geographical location information of the user, more effective advertisement display becomes possible. Part or all of the above-described processing in the display unit may be performed using, for example, AI, or may be performed without using AI. For example, the display unit can input geographical location information data of the user to a generative AI and cause the generative AI to execute display of a highly relevant advertisement. Specifically, the present display unit uses hyper-local targeting technology. When the present display unit detects from GPS information that the user has entered a specific geofence (e.g., within a radius of 500 m of a specific shopping mall), the present display unit triggers an advertisement campaign limited to that area. Furthermore, in cooperation with a weather API, advertisement content is switched according to the weather (rain, intense heat, etc.) of the current location (e.g., advertisement for ice cream or cold beverage on a hot day, advertisement for delivery service on a rainy day). Also, predictive distribution is performed in which a “commuting route” or a “living sphere” is learned from a movement history of the user, and an advertisement of a store on the route is displayed at a timing when the user approaches the place.

The display unit can analyze a social media activity of the user and display a related advertisement when displaying the advertisement. For example, the display unit displays an advertisement related to a product or service shared by the user on social media. Also, the display unit can display an advertisement related to a brand or service followed by the user on social media. Furthermore, the display unit can analyze a trend of a community in which the user participates on social media and display a related advertisement. Thereby, by analyzing the social media activity of the user, a related advertisement can be displayed. Part or all of the above-described processing in the display unit may be performed using, for example, AI, or may be performed without using AI. For example, the display unit can input social media activity data of the user to a generative AI and cause the generative AI to execute display of a related advertisement. Specifically, the present display unit performs social graph mining and scores potential brand affinity of the user. The present display unit analyzes image feature amounts and text topics of posts “liked” by the user, and selects an advertisement of a brand having a world view or values similar thereto (Look-alike Modeling). For example, an advertisement for natural food or eco-goods is displayed to a user who frequently reacts to posts regarding organic food. Also, a “social endorsement advertisement” displaying an advertisement of a product purchased or recommended by a friend of the user is generated, and processing of increasing reliability and a click rate of the advertisement is performed by adding a wording such as “Your friend Mr./Ms. XX also uses it.”

The display unit can analyze an emotion of the user and adjust a display method of an advertisement based on the analyzed emotion of the user. For example, when the user feels stress, the display unit displays a simple and highly visible advertisement. Also, when the user is relaxed, the display unit can display an advertisement including detailed information. Furthermore, when the user is in a hurry, the display unit can display an advertisement focusing on main points. Thereby, by adjusting the display method of the advertisement according to the emotion of the user, more effective advertisement display becomes possible. The estimation of the emotion is realized using an emotion estimation function using, for example, an emotion engine or generative AI. The generative AI is a text generation AI (for example, LLM), a multimodal generation AI, or the like, but is not limited to such examples. Part or all of the above-described processing in the display unit may be performed using AI, for example, or may be performed without using AI. For example, the display unit can input emotion data of the user into the generative AI and cause the generative AI to execute adjustment of the display method of the advertisement. Specifically, in addition to the content described in 0098, the present display unit has a dynamic conversion function of an advertisement format itself. When the user is “in a hurry”, the present display unit automatically converts a video advertisement into a still image thumbnail and a short text summary to display it, thereby instantly transmitting information while saving data traffic. Also, when the user is “relaxed”, the present display unit adopts a parallax effect that expands in conjunction with scrolling or an interactive advertisement format in which a story progresses by a swipe operation, thereby performing a presentation to extend a stay time. This format conversion is executed in real time immediately after receiving an emotion estimation result by client-side rendering logic using HTML5, CSS3, and JavaScript.

The system according to the embodiment is not limited to the above-described examples, and various modifications are possible, for example, as follows. Specifically, the present system can adopt not only a monolithic architecture but also a microservices architecture to deploy each function (receiving, proposal, acquisition, accumulation, identification, display) as an independent container and manage them with an orchestration tool such as Kubernetes. Also, it is possible to adopt an edge-cloud cooperation configuration in which part of processing (for example, emotion estimation requiring real-time performance or pre-processing of images) is executed on a user terminal (edge device) side, and processing with high calculation load (inference by a large-scale language model or large-scale database search) is executed on a cloud server side. Furthermore, introducing blockchain technology to prevent falsification of health data and allergy information of the user and constructing a token economy (reward system) for data provision are also modifications included in the technical scope of the present invention.

The proposal unit can also propose a recipe based on a meal time zone of the user. For example, in a breakfast time zone, the proposal unit proposes a recipe suitable for energy supply, and in a lunch time zone, proposes a recipe with a good nutritional balance. Also, in a dinner time zone, the proposal unit can propose a recipe that is easy to digest. Furthermore, the proposal unit can propose a recipe considering expiration dates of ingredients based on the meal time zone of the user. Thereby, it is possible to propose an optimal recipe according to the meal time zone of the user. Specifically, the present proposal unit implements a Chrono-nutrition Model based on circadian rhythm and nutrition science. The present proposal unit takes not only the current time but also a lifestyle rhythm (wake-up time, bedtime) of the user as input, and estimates a phase of a biological clock. For example, at breakfast, a score of a recipe containing carbohydrates with a high GI value and protein is set high to raise body temperature and activate the brain. At dinner, a cooking method with less digestive burden or a recipe containing ingredients that help melatonin production is recommended to improve sleep quality. Also, in cooperation with an ingredient management system in a refrigerator, when there is an ingredient whose expiration date is approaching (for example, milk with “3 hours left until the expiration date”), logic to preferentially propose a recipe (such as stew or gratin) capable of consuming a large amount of the ingredient as a meal in that time zone is executed, contributing to food loss reduction.

The display unit can also display a related coupon based on a purchase history of the user. For example, the display unit displays a coupon related to a product purchased by the user in the past to encourage repurchase. Also, the display unit can display a coupon for a new product in which the user is likely to be interested from the purchase history of the user. Furthermore, the display unit can analyze the purchase history of the user and select and display the most effective coupon. Thereby, an effective coupon can be displayed based on the purchase history of the user. Specifically, the present display unit uses a Propensity to Coupon Model. This model takes past purchase data (RFM analysis: Recency, Frequency, Monetary) of the user and coupon usage history as input, and predicts a probability that each user reacts to a specific coupon. The present display unit performs “targeting issuance” that displays a coupon with a high discount rate only to a user with a high predicted probability, thereby maximizing return on investment (ROI) of promotion costs. Also, the display unit performs association analysis (market basket analysis) and makes a cross-selling proposal that displays a coupon for edamame to a user with a beer purchase history based on a rule such as “people who buy beer tend to buy edamame as well”. Furthermore, the display unit also has a function of dynamically generating a display format of the coupon (barcode, QR code, for NFC touch) according to a device used by the user or a POS system of a store.

The receiving unit can analyze an emotion of the user and adjust an order of information to be input based on the analyzed emotion of the user. For example, when the user feels stress, the receiving unit makes the user input important information first and postpones other information. Also, when the user is relaxed, the receiving unit can make the user input detailed information first. Furthermore, when the user is in a hurry, the receiving unit can make the user input only the most important information first. Thereby, by adjusting the order of information to be input according to the emotion of the user, more appropriate information can be input. Specifically, the present receiving unit executes an algorithm for dynamically reconstructing a State Transition Graph of an input form. In a normal state, the transition is linear as basic information ->detailed information->confirmation, but when the user is in a “stress” or “impatience” state, the present receiving unit changes a graph structure and generates a Shortest Path passing only through essential nodes (important information input screens) to guide the user. At this time, omitted nodes (detailed information input screens) are saved in the background as a “task list” for input at a later date. Also, not only the order of input items but also the number of items displayed on one screen (chunk size) is adjusted according to the emotion to optimize cognitive load. For example, a wizard format of One Question Per Screen is adopted at the time of stress, and a list format with high listability is adopted at the time of relaxation.

The receiving unit can analyze a past input history of the user and provide an optimal input interface. For example, the receiving unit automatically displays allergy information or food preferences frequently input by the user in the past as candidates. Also, the receiving unit can preferentially propose an input method (voice, text, etc.) used by the user in the past. Furthermore, the receiving unit can predict and propose information used in a specific time zone from the past input history of the user. Thereby, by analyzing the past input history, an optimal input interface can be provided to the user. Specifically, in addition to the content described in 0069, the present receiving unit automatically generates a UI/UX setting profile for each user by machine learning. The present receiving unit analyzes detailed operation logs such as at which input field the user left in the past, which button was erroneously tapped, and with which input method the number of corrections was large. For example, for a user with many erroneous inputs in flick input, a size of a keyboard is automatically enlarged, or voice input is set as a default. Also, when scrolling occurs every time a specific ingredient is input, a layout change is performed to fix the ingredient at the top of a list. These optimizations are performed by a reinforcement learning agent, and learning proceeds with minimizing a Time to Completion of the user as a reward.

The receiving unit can customize an input item based on a current health condition or a lifestyle habit of the user when information is input. For example, when the user inputs a result of a health checkup, the receiving unit adds a necessary nutrient to the input item based on the result. Also, when the user inputs an amount of exercise, the receiving unit can add a calorie intake amount corresponding to the amount of exercise to the input item. Furthermore, when the user inputs a specific lifestyle habit (for example, vegetarian), the receiving unit can customize the input item based on the habit. Thereby, by customizing the input item based on the health condition or the lifestyle habit of the user, more appropriate information can be input. Specifically, in addition to the content described in 0070, the present receiving unit includes a data importer compatible with a medical data exchange standard (HL7 FHIR, etc.) and supports data import from an electronic medical record or a health management application. Based on imported structured data, the present receiving unit rewrites metadata of an input form. For example, when a diagnosis code of diabetes is included, detailed setting items regarding “carbohydrate intake” or “GI value restriction” are automatically expanded. Also, based on real-time activity amount data (current calorie consumption) acquired from a wearable device, an item for inputting (or confirming) “today's additional ingestible calories” is dynamically displayed, providing an interface that supports health management behavior of the user on the spot.

The receiving unit can estimate an emotion of the user and determine a priority order of information to be input based on the estimated emotion of the user. For example, when the user feels stress, the receiving unit makes the user preferentially input important information and postpones other information. Also, when the user is relaxed, the receiving unit can make the user preferentially input detailed information. Furthermore, when the user is in a hurry, the receiving unit can make the user preferentially input only the most important information. Thereby, by determining the priority order of information to be input according to the emotion of the user, more appropriate information can be input. Specifically, in addition to the content described in 0071, the present receiving unit has logic to dynamically calculate an “importance score” of an input item. This score is calculated by a product of a mandatory requirement of the system (static weight) and an emotion context of the user (dynamic coefficient). For example, at the time of stress, a coefficient of a “system mandatory item” is increased, and a coefficient of an “optional item” is set to zero. Furthermore, the present receiving unit applies a UI design principle based on Gestalt psychology, arranges an item with a high priority order in the center of a screen or at a destination of a line of sight, and emphasizes it with color or size (dynamic change of Visual Hierarchy). Thereby, the user is guided to input from important information unconsciously, and can complete a task without feeling an emotional burden.

The receiving unit can preferentially display a highly relevant input item based on geographical location information of the user when information is input. For example, when the user lives in a specific area, the receiving unit preferentially displays an ingredient frequently consumed in that area as the input item. Also, when the user is traveling, the receiving unit can preferentially display a specialty product or an ingredient of a travel destination as the input item. Furthermore, when the user uses a supermarket in a specific area, the receiving unit can preferentially display an ingredient sold at the supermarket as the input item. Thereby, by preferentially displaying a highly relevant input item based on the geographical location information of the user, more appropriate information can be input. Specifically, in addition to the content described in 0072, the present receiving unit performs cross-reference with weather data. The input item is adjusted based on current weather (temperature, humidity) of the area identified from the location information. For example, when the user is in a hot area, “cold noodles” or “summer vegetables” are displayed at the top of input candidates. Also, in cooperation with an in-store map (indoor map) of a supermarket, when it is detected by a beacon or the like that the user is in a “fresh fish corner” in the store, context-aware input support is realized in which an input item related to fresh fish or a recipe search window is immediately pop-up displayed.

The receiving unit can analyze a social media activity of the user and propose a related input item when information is input. For example, the receiving unit analyzes a photo of a meal shared by the user on social media and proposes a related ingredient as the input item. Also, the receiving unit can analyze a recipe of a cooking account followed by the user on social media and propose a related ingredient as the input item. Furthermore, the receiving unit can analyze a trend of a cooking community in which the user participates on social media and propose a related ingredient as the input item. Thereby, by analyzing the social media activity of the user, a related input item can be proposed. Specifically, in addition to the content described in 0073, the present receiving unit uses an unsupervised learning model (k-means method, etc.) that identifies a “food preference cluster” of the user. Based on a feature vector extracted from SNS activity, the user is classified into a cluster such as “health-oriented”, “hearty eater”, or “sweets lover”. The present receiving unit proposes an item frequently input by other members of the belonging cluster as a “recommendation” (User-based Collaborative Filtering). For example, for a user belonging to the “sweets lover” cluster, an input item for confectionery ingredients or fruits is expanded by default, thereby saving time and effort for input and satisfying a potential need of the user in advance.

The proposal unit can analyze an emotion of the user and adjust a presentation method of a recipe based on the analyzed emotion of the user. For example, when the user feels stress, the proposal unit proposes a simple and easy-to-understand recipe. Also, when the user is relaxed, the proposal unit can propose a recipe including a detailed procedure and an arrangement method. Furthermore, when the user is in a hurry, the proposal unit can propose a recipe that can be made in a short time. Thereby, by adjusting the presentation method of the recipe according to the emotion of the user, a more appropriate recipe can be proposed. Specifically, in addition to the content described in 0074, the present proposal unit has a multimedia conversion function of recipe content. When the user feels “stress” or “fatigue”, in order to reduce a burden of reading text, a mode is switched to a mode in which a procedure of the recipe is read out by an automatically generated voice guide (Text-to-Speech) or a summarized short video clip (Video Summary) is played. Also, in cooperation with a smart speaker or a smart display, an interface capable of confirming a cooking procedure hands-free is provided. Conversely, when the user is “relaxed”, a high-resolution completed photo or a column text explaining a cooking tip is displayed to perform presentation that enhances value as reading material.

The proposal unit can analyze a past meal history of the user and select an optimal recipe when proposing a recipe. For example, based on a dish that the user liked to eat in the past, the proposal unit proposes a similar recipe. Also, based on a dish that the user avoided in the past, the proposal unit can propose a recipe that does not include it. Furthermore, the proposal unit can propose a recipe considering a nutritional balance from the past meal history of the user. Thereby, by analyzing the past meal history of the user, an optimal recipe can be proposed. Specifically, in addition to the content described in 0075, the present proposal unit uses a Recurrent Neural Network (RNN) that models a long-term change in taste. Since human taste and preference change depending on season, age, and environment, not only a recent history but also a history of the same period one year ago and a long-term trend (e.g., gradually preferring a lighter taste) are learned. The present proposal unit predicts a recipe that the current user will feel most delicious in consideration of this “taste drift”. Also, an algorithm for detecting boredom is incorporated, and when similar recipes continue, control is performed to dare to propose an “exploratory recipe (Serendipity)” with a slightly different tendency to prevent getting into a rut.

The flow of processing of Example of the Embodiment will be briefly described below. Specifically, a series of processing in the present system is designed based on an event-driven architecture, and each step is executed asynchronously and in parallel with a user action or an update of external data as a trigger. Each step shown below is a logical processing unit, and in actual implementation, it is configured as a data pipeline via an API call between microservices or a message queue (Kafka, etc.).

Step 1: The receiving unit receives information such as allergy information, food preferences, and an amount of exercise of the user. The information input by the user includes, for example, an allergy to a specific food, likes and dislikes, a daily amount of exercise, and the like. The receiving unit enables the user to input information through an application on a smartphone or a tablet. Step 2: The proposal unit analyzes the information received by the receiving unit and proposes an appropriate recipe. The proposal unit uses AI to generate a healthy and balanced recipe in consideration of the allergy information, food preferences, amount of exercise, and the like. For example, the proposal unit proposes a recipe that excludes a food having an allergy and considers calories according to the amount of exercise. Step 3: The acquisition unit acquires special sale information of a nearby supermarket. The acquisition unit collects and analyzes the special sale information of the nearby supermarket through the Internet. For example, the acquisition unit analyzes the special sale information and generates an economical and highly nutritious recipe. Step 4: The accumulation unit accumulates ingredient list images of consumed food. The accumulation unit uploads an ingredient list image of a food consumed by the user to the application and analyzes its ingredients. The ingredient list image is stored in a database and used to identify an ingredient that may cause an allergy. Step 5: The identification unit identifies which ingredient is suspected when an allergic symptom occurs. The identification unit analyzes the ingredient list image using AI and identifies an ingredient that may cause the allergy. Based on the ingredient list image of the food consumed by the user, the ingredient causing the allergy is identified. Specifically, in Step 1, the receiving unit performs validation and normalization of input data and issues a transaction to update a user profile DB. In Step 2, the proposal unit drives an inference engine using the updated profile and a recipe DB, generates a recommended recipe list, and stores it in a cache server. In Step 3, the acquisition unit operates a crawler in the background, acquires and structures the special sale information, and reflects it in recipe generation logic in real time. In Step 4, the accumulation unit receives an upload of image data and performs storage in a distributed storage and queuing of an OCR analysis job. In Step 5, the identification unit receives a trigger (symptom report) from the user, collates accumulated analyzed data with an allergen knowledge base, calculates a probability ranking of a causative substance, and push-notifies it to a user terminal. These steps are executed at high speed and scalably while maintaining consistency of the entire system.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of a plurality of elements including the receiving unit, the proposal unit, the acquisition unit, the accumulation unit, the identification unit, and the display unit described above is implemented by, for example, at least one of a smart device 14 and a data processing device 12. For example, the receiving unit is implemented by a control unit 46A of the smart device 14, and enables a user to input information through an application on a smartphone or a tablet. The proposal unit is implemented by, for example, a specific processing unit 290 of the data processing device 12, and generates a recipe considering allergy information, food preferences, an amount of exercise, and the like using AI. The acquisition unit is implemented by, for example, the specific processing unit 290 of the data processing device 12, and collects and analyzes special sale information of a nearby supermarket through the Internet. The accumulation unit is implemented by, for example, the control unit 46A of the smart device 14, uploads ingredient list images of food consumed by the user to the application, and analyzes ingredients thereof. The identification unit is implemented by, for example, the specific processing unit 290 of the data processing device 12, analyzes the ingredient list images, and identifies an ingredient causing an allergy. The display unit is implemented by, for example, the control unit 46A of the smart device 14, and displays an advertisement at the bottom of a recipe page. The correspondence relationship between each unit and the device or the control unit is not limited to the above-described example, and various modifications are possible.

### Second Embodiment

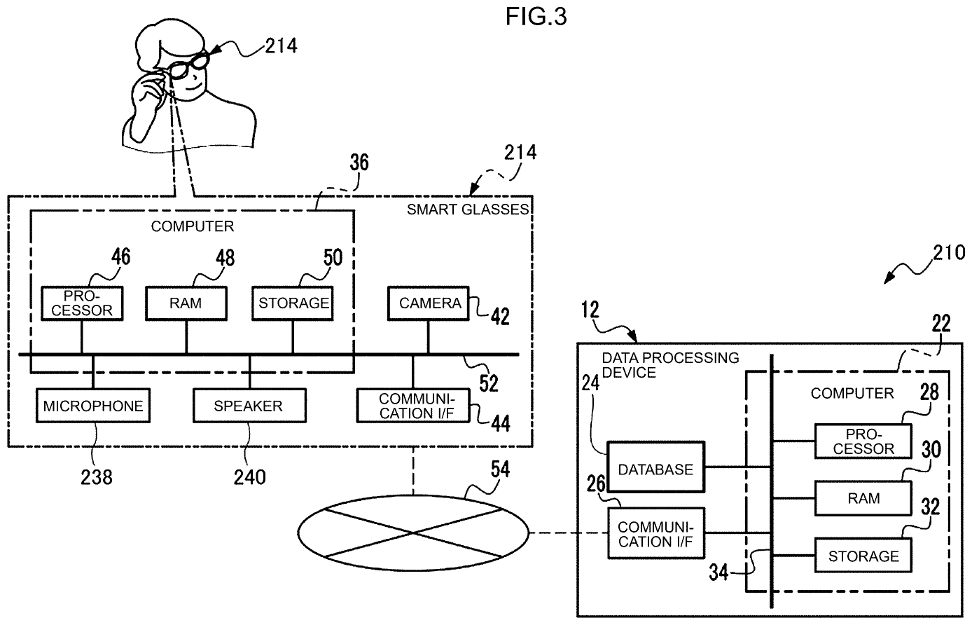

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

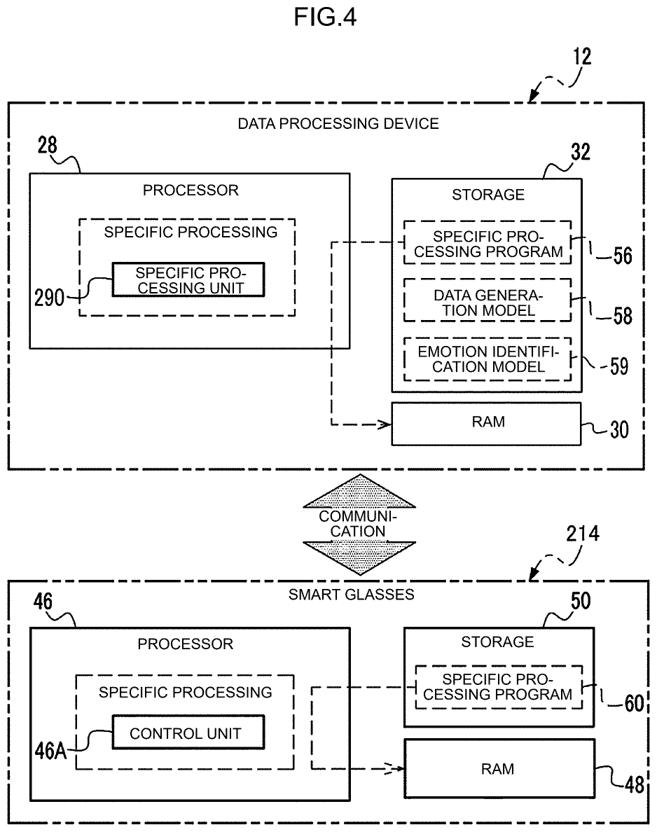

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of a plurality of elements including the receiving unit, the proposal unit, the acquisition unit, the accumulation unit, the identification unit, and the display unit described above is implemented by, for example, at least one of smart glasses 214 and a data processing device 12. For example, the receiving unit is implemented by a control unit 46A of the smart glasses 214, and enables a user to input information through the smart glasses. The proposal unit is implemented by, for example, a specific processing unit 290 of the data processing device 12, and generates a recipe considering allergy information, food preferences, an amount of exercise, and the like using AI. The acquisition unit is implemented by, for example, the specific processing unit 290 of the data processing device 12, and collects and analyzes special sale information of a nearby supermarket through the Internet. The accumulation unit is implemented by, for example, the control unit 46A of the smart glasses 214, uploads ingredient list images of food consumed by the user, and analyzes ingredients thereof. The identification unit is implemented by, for example, the specific processing unit 290 of the data processing device 12, analyzes the ingredient list images, and identifies an ingredient causing an allergy. The display unit is implemented by, for example, the control unit 46A of the smart glasses 214, and displays an advertisement at the bottom of a recipe page. The correspondence relationship between each unit and the device or the control unit is not limited to the above-described example, and various modifications are possible.

### Third Embodiment

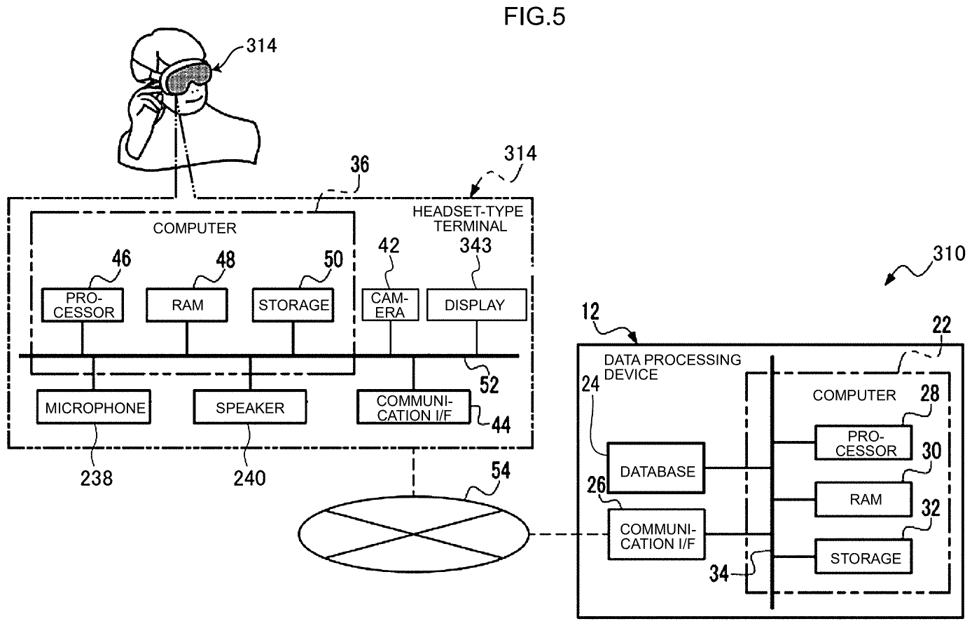

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

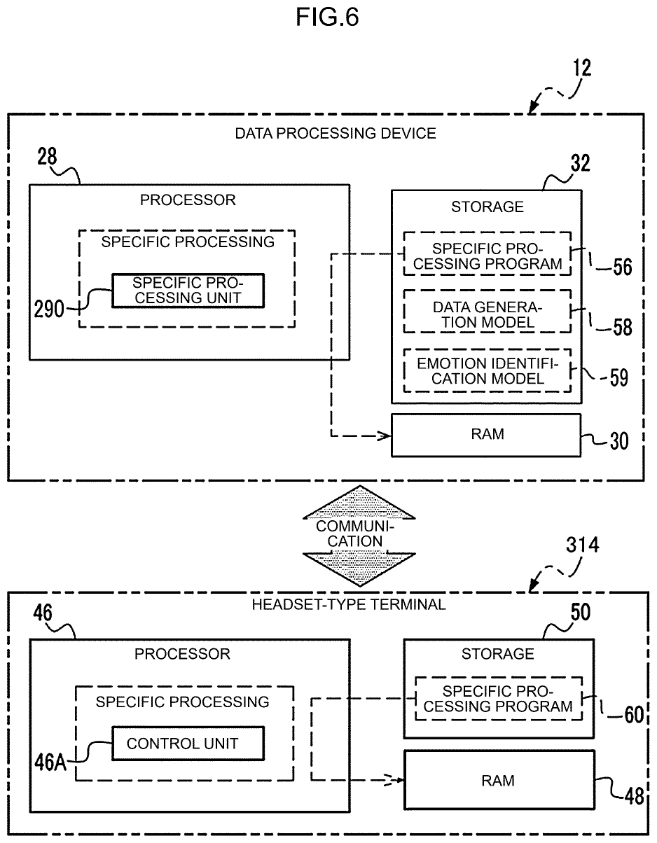

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of a plurality of elements including the receiving unit, the proposal unit, the acquisition unit, the accumulation unit, the identification unit, and the display unit described above is implemented by, for example, at least one of a headset-type terminal 314 and a data processing device 12. For example, the receiving unit is implemented by a control unit 46A of the headset-type terminal 314, and enables a user to input information through the headset. The proposal unit is implemented by, for example, a specific processing unit 290 of the data processing device 12, and generates a recipe considering allergy information, food preferences, an amount of exercise, and the like using AI. The acquisition unit is implemented by, for example, the specific processing unit 290 of the data processing device 12, and collects and analyzes special sale information of a nearby supermarket through the Internet. The accumulation unit is implemented by, for example, the control unit 46A of the headset-type terminal 314, uploads ingredient list images of food consumed by the user, and analyzes ingredients thereof. The identification unit is implemented by, for example, the specific processing unit 290 of the data processing device 12, analyzes the ingredient list images, and identifies an ingredient causing an allergy. The display unit is implemented by, for example, the control unit 46A of the headset-type terminal 314, and displays an advertisement at the bottom of a recipe page. The correspondence relationship between each unit and the device or the control unit is not limited to the above-described example, and various modifications are possible.

### Fourth Embodiment

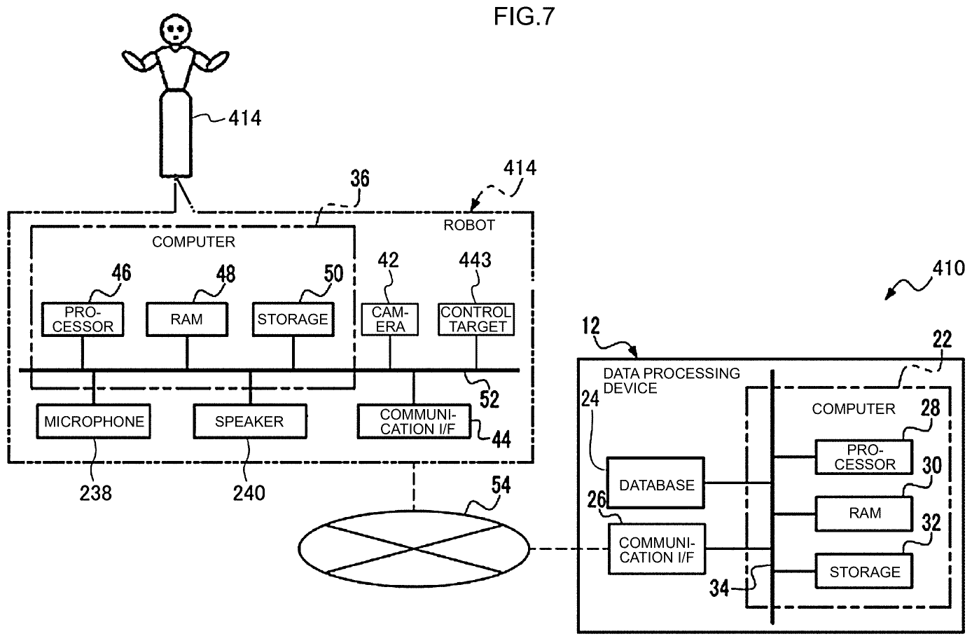

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

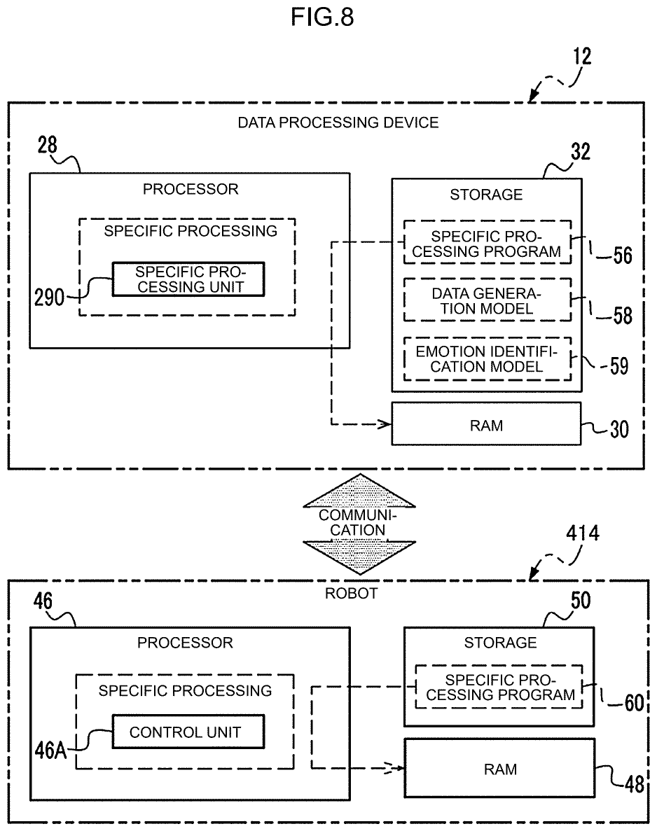

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of a plurality of elements including the receiving unit, the proposal unit, the acquisition unit, the accumulation unit, the identification unit, and the display unit described above is implemented by, for example, at least one of a robot 414 and a data processing device 12. For example, the receiving unit is implemented by a control unit 46A of the robot 414, and enables a user to input information through the robot. The proposal unit is implemented by, for example, a specific processing unit 290 of the data processing device 12, and generates a recipe considering allergy information, food preferences, an amount of exercise, and the like using AI. The acquisition unit is implemented by, for example, the specific processing unit 290 of the data processing device 12, and collects and analyzes special sale information of a nearby supermarket through the Internet. The accumulation unit is implemented by, for example, the control unit 46A of the robot 414, uploads ingredient list images of food consumed by the user, and analyzes ingredients thereof. The identification unit is implemented by, for example, the specific processing unit 290 of the data processing device 12, analyzes the ingredient list images, and identifies an ingredient causing an allergy. The display unit is implemented by, for example, the control unit 46A of the robot 414, and displays an advertisement at the bottom of a recipe page. The correspondence relationship between each unit and the device or the control unit is not limited to the above-described example, and various modifications are possible.

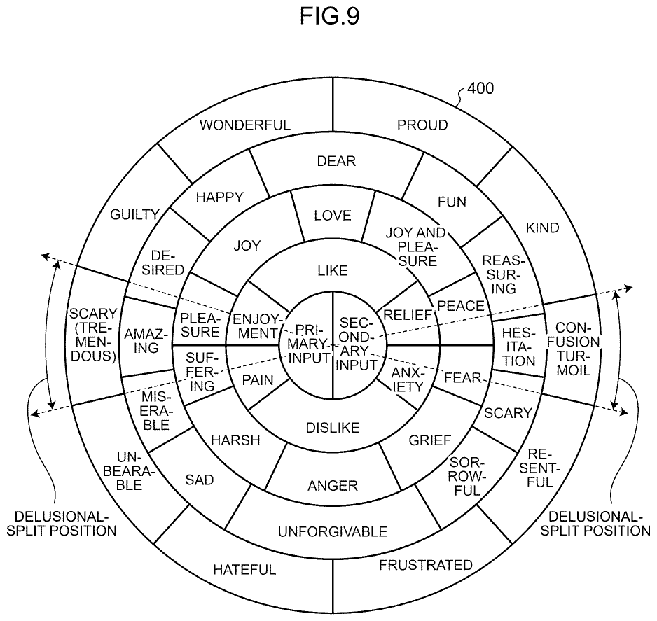

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

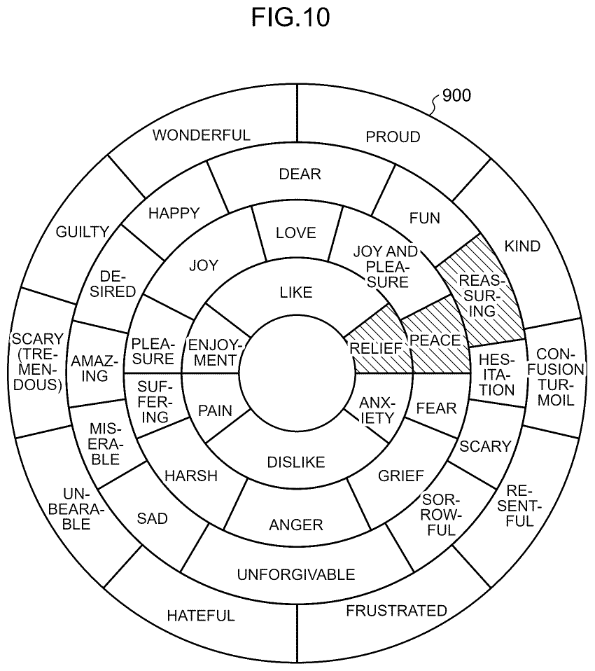

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a receiving unit configured to receive allergy information, food preferences, or information on an amount of exercise of a user; a proposal unit configured to analyze the information received by the receiving unit and propose a recipe; an acquisition unit configured to acquire special sale information of a nearby supermarket; an accumulation unit configured to accumulate ingredient list images of consumed food; and an identification unit configured to identify which ingredient is suspected when an allergic symptom occurs.

(Supplementary Note 2) The system according to Supplementary Note 1, further comprising a display unit configured to display an advertisement.

(Supplementary Note 3) The system according to Supplementary Note 2, wherein the display unit displays the advertisement based on an interest or concern of the user.

(Supplementary Note 4) The system according to Supplementary Note 1, wherein the receiving unit analyzes an emotion of the user and adjusts an input method of information based on the analyzed emotion of the user.

(Supplementary Note 5) The system according to Supplementary Note 1, wherein the receiving unit analyzes a past input history of the user and provides an input interface.

(Supplementary Note 6) The system according to Supplementary Note 1, wherein the receiving unit customizes an input item based on a current health condition or a lifestyle habit of the user when information is input.

(Supplementary Note 7) The system according to Supplementary Note 1, wherein the receiving unit estimates an emotion of the user and determines a priority order of information to be input based on the estimated emotion of the user.

(Supplementary Note 8) The system according to Supplementary Note 1, wherein the receiving unit preferentially displays a highly relevant input item based on geographical location information of the user when information is input.

(Supplementary Note 9) The system according to Supplementary Note 1, wherein the receiving unit analyzes a social media activity of the user and proposes a related input item when information is input.

(Supplementary Note 10) The system according to Supplementary Note 1, wherein the proposal unit analyzes an emotion of the user and adjusts a presentation method of the recipe based on the analyzed emotion of the user.

(Supplementary Note 11) The system according to Supplementary Note 1, wherein the proposal unit analyzes a past meal history of the user and selects a recipe when proposing the recipe.

(Supplementary Note 12) The system according to Supplementary Note 1, wherein the proposal unit customizes the recipe based on a current health condition or a lifestyle habit of the user when proposing the recipe.

(Supplementary Note 13) The system according to Supplementary Note 1, wherein the proposal unit analyzes an emotion of the user and determines a priority order of recipes based on the analyzed emotion of the user.

(Supplementary Note 14) The system according to Supplementary Note 1, wherein the proposal unit preferentially proposes a highly relevant recipe based on geographical location information of the user when proposing the recipe.

(Supplementary Note 15) The system according to Supplementary Note 1, wherein the proposal unit analyzes a social media activity of the user and proposes a related recipe when proposing the recipe.

(Supplementary Note 16) The system according to Supplementary Note 1, wherein the acquisition unit analyzes an emotion of the user and adjusts an acquisition timing of the special sale information based on the analyzed emotion of the user.

(Supplementary Note 17) The system according to Supplementary Note 1, wherein the acquisition unit analyzes past special sale information of the nearby supermarket and selects an acquisition method.

(Supplementary Note 18) The system according to Supplementary Note 1, wherein the acquisition unit performs filtering based on a current purchase history or a field of interest of the user when acquiring the special sale information.

(Supplementary Note 19) The system according to Supplementary Note 1, wherein the acquisition unit analyzes an emotion of the user and determines a priority order of the special sale information to be acquired based on the analyzed emotion of the user.

(Supplementary Note 20) The system according to Supplementary Note 1, wherein the acquisition unit preferentially acquires highly relevant information based on geographical location information of the user when acquiring the special sale information.

(Supplementary Note 21) The system according to Supplementary Note 1, wherein the acquisition unit analyzes a social media activity of the user and acquires related information when acquiring the special sale information.

(Supplementary Note 22) The system according to Supplementary Note 1, wherein the accumulation unit analyzes an emotion of the user and adjusts an accumulation method of the ingredient list images based on the analyzed emotion of the user.

(Supplementary Note 23) The system according to Supplementary Note 1, wherein the accumulation unit analyzes a past consumption history of the user and selects an accumulation method when accumulating the ingredient list images.

(Supplementary Note 24) The system according to Supplementary Note 1, wherein the accumulation unit customizes an accumulation item based on a current health condition or a lifestyle habit of the user when accumulating the ingredient list images.

(Supplementary Note 25) The system according to Supplementary Note 1, wherein the accumulation unit analyzes an emotion of the user and determines a priority order of the ingredient list images to be accumulated based on the analyzed emotion of the user.

(Supplementary Note 26) The system according to Supplementary Note 1, wherein the accumulation unit preferentially accumulates a highly relevant image based on geographical location information of the user when accumulating the ingredient list images.

(Supplementary Note 27) The system according to Supplementary Note 1, wherein the accumulation unit analyzes a social media activity of the user and accumulates a related image when accumulating the ingredient list images.

(Supplementary Note 28) The system according to Supplementary Note 1, wherein the identification unit analyzes an emotion of the user and adjusts an identification method of an allergic ingredient based on the analyzed emotion of the user.

(Supplementary Note 29) The system according to Supplementary Note 1, wherein the identification unit analyzes a past allergy history of the user and selects an identification method when identifying the allergic ingredient.

(Supplementary Note 30) The system according to Supplementary Note 1, wherein the identification unit customizes an identification item based on a current health condition or a lifestyle habit of the user when identifying the allergic ingredient.

(Supplementary Note 31) The system according to Supplementary Note 1, wherein the identification unit analyzes an emotion of the user and determines a priority order of the allergic ingredient to be identified based on the analyzed emotion of the user.

(Supplementary Note 32) The system according to Supplementary Note 1, wherein the identification unit preferentially identifies a highly relevant ingredient based on geographical location information of the user when identifying the allergic ingredient.

(Supplementary Note 33) The system according to Supplementary Note 1, wherein the identification unit analyzes a social media activity of the user and identifies a related ingredient when identifying the allergic ingredient.

(Supplementary Note 34) The system according to Supplementary Note 2, wherein the display unit analyzes an emotion of the user and adjusts a display method of the advertisement based on the analyzed emotion of the user.

(Supplementary Note 35) The system according to Supplementary Note 2, wherein the display unit analyzes a past browsing history of the user and selects the advertisement when displaying the advertisement.

(Supplementary Note 36) The system according to Supplementary Note 2, wherein the display unit customizes the advertisement based on a current interest or concern of the user when displaying the advertisement.

(Supplementary Note 37) The system according to Supplementary Note 2, wherein the display unit analyzes an emotion of the user and determines a priority order of the advertisement based on the analyzed emotion of the user.

(Supplementary Note 38) The system according to Supplementary Note 2, wherein the display unit preferentially displays a highly relevant advertisement based on geographical location information of the user when displaying the advertisement.

(Supplementary Note 39) The system according to Supplementary Note 2, wherein the display unit analyzes a social media activity of the user and displays a related advertisement when displaying the advertisement.

What is claimed is:

1. A system comprising:
circuitry configured to:
receive, from a client terminal via a communication interface and a packet-switched network, user attribute data comprising at least one of preference data, restriction data, or activity data;
extract a feature vector from the user attribute data by inputting the user attribute data into a feature extraction model comprising at least one of a convolutional neural network or a Transformer-based model;
calculate a similarity metric between the extracted feature vector and reference feature vectors stored in a database to identify a matching record;
generate, using a data generation model comprising a neural network obtained by deep learning, inference data based on the identified matching record; and
transmit the inference data to the client terminal via the communication interface and the packet-switched network.

2. The system according to claim 1, wherein the user attribute data further comprises sensor data received from a wearable device of the user, the sensor data comprising at least one of step count data, heart rate data, or calorie consumption data.

3. The system according to claim 1, wherein the preference data comprises a preference feature vector representing at least one of a food preference, a taste preference, or a dietary restriction, and wherein the restriction data comprises a one-hot vector indicating items to be excluded from the inference data.

4. The system according to claim 1, wherein the circuitry is further configured to estimate an emotion of the user by applying an emotion identification model to at least one of voice data, a face image, or text input received from the client terminal, and to adjust a presentation format of the inference data based on the estimated emotion.

5. The system according to claim 4, wherein the circuitry is further configured to simplify the presentation format of the inference data when the estimated emotion indicates stress, and to provide a detailed presentation format when the estimated emotion indicates relaxation.

6. The system according to claim 1, wherein the similarity metric comprises at least one of a cosine similarity or a Euclidean distance, and wherein the circuitry is configured to perform an approximate nearest neighbor search to identify the matching record from the reference feature vectors.

7. The system according to claim 1, wherein the data generation model comprises a large language model adopting a Transformer architecture, and wherein generating the inference data comprises inputting the identified matching record and the extracted feature vector as a prompt to the large language model to generate a natural-language output.

8. The system according to claim 1, wherein the circuitry is further configured to receive image data captured by a camera of the client terminal, extract text data from the image data using optical character recognition, and store the extracted text data in the database as structured data associated with a timestamp.

9. The system according to claim 8, wherein the circuitry is further configured to analyze the extracted text data to identify ingredient information, compare the ingredient information with restriction data stored in the database, and generate an alert when a match is detected.

10. The system according to claim 1, wherein the circuitry is further configured to acquire external data from an external server via the communication interface, extract structured information from the external data using a natural language processing model, and store the structured information in the database.

11. The system according to claim 10, wherein the external data comprises promotion data associated with a geographic region, and wherein the circuitry is further configured to filter the promotion data based on geographic location information of the user received from the client terminal.

12. The system according to claim 1, wherein the circuitry is further configured to analyze a past input history of the user stored in the database using a recurrent neural network or a long short-term memory network to predict an input item, and to provide the predicted input item as an autocomplete candidate to the client terminal.

13. The system according to claim 1, wherein the circuitry is further configured to receive health profile data of the user comprising at least one of a body mass index, a blood pressure value, or a blood glucose level, and to adjust a parameter of the data generation model based on the health profile data to generate the inference data adapted to a health condition of the user.

14. The system according to claim 1, wherein the circuitry is further configured to analyze social media activity data of the user received via an application programming interface, extract interest information using a natural language processing model comprising at least one of BERT or a Transformer-based model, and incorporate the extracted interest information into the feature vector.

15. The system according to claim 1, wherein the circuitry is further configured to determine a priority of generating the inference data based on an emotion of the user estimated using an emotion identification model, such that when the estimated emotion indicates urgency, essential information is prioritized in the inference data.

16. The system according to claim 1, wherein the circuitry is further configured to generate advertisement display data based on an interest vector of the user calculated from browsing history data stored in the database, and to transmit the advertisement display data to the client terminal together with the inference data.

17. The system according to claim 1, wherein the circuitry is further configured to search a vector database using a retrieval-augmented generation architecture to acquire reference documents related to the user attribute data, and to input the acquired reference documents together with the extracted feature vector into the data generation model to generate the inference data.

18. A system comprising:
a communication interface configured to communicate, via a packet-switched network conforming to at least one of a 5G, Wi-Fi, or Bluetooth communication standard, with a client terminal comprising a camera having a CMOS image sensor, a touch panel, a microphone, a speaker, and a display;
a processor comprising at least one of a CPU, a GPU, or a TPU;
a random-access memory;
a memory storing a data generation model comprising a large language model adopting a Transformer architecture obtained by deep learning on a neural network, and an emotion identification model;
a database storing reference feature vectors and user profile data; and
circuitry configured to:
receive, from the client terminal via the communication interface, user attribute data comprising at least one of preference data indicating a food preference, restriction data indicating an allergy or dietary restriction, or activity data indicating an amount of exercise;
extract a feature vector from the user attribute data by inputting the user attribute data into a feature extraction model;
calculate at least one of a cosine similarity or a Euclidean distance between the extracted feature vector and the reference feature vectors stored in the database to identify a matching record;
estimate an emotion of the user by applying the emotion identification model to at least one of voice data captured by the microphone or image data captured by the camera;
generate, using the data generation model, inference data comprising a recommendation adapted to the user attribute data and the estimated emotion; and
transmit the inference data to the client terminal via the communication interface, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to generate the inference data based on a prompt comprising the identified matching record and the estimated emotion without additional instructions.

20. A method performed by circuitry of a data processing system comprising a processor, a random-access memory, a memory storing a data generation model obtained by deep learning on a neural network and an emotion identification model, a database storing reference feature vectors, and a communication interface, the method comprising:
receiving, from a client terminal via the communication interface and a packet-switched network, user attribute data comprising at least one of preference data, restriction data, or activity data;
extracting a feature vector from the user attribute data by inputting the user attribute data into a feature extraction model comprising at least one of a convolutional neural network or a Transformer-based model;
calculating a similarity metric between the extracted feature vector and the reference feature vectors stored in the database to identify a matching record;
generating, using the data generation model, inference data based on the identified matching record; and
transmitting the inference data to the client terminal via the communication interface and the packet-switched network.
